SEQUENCE LISTING (26 sequences)

SEQ ID NO: 1 - DNA, 33 bp, Artificial Sequence
  primer
ttcacacaaagtaaggggcttttcgttatgatg

SEQ ID NO: 2 - DNA, 27 bp, Artificial Sequence
  primer
aggtgatttacgcggcttcaactttcc

SEQ ID NO: 3 - DNA, 35 bp, Artificial Sequence
  primer
cgactctagaggatctctagacgctcaagttagta

SEQ ID NO: 4 - DNA, 27 bp, Artificial Sequence
  primer
cttactttgtgtgaaattgttatccgc

SEQ ID NO: 5 - DNA, 33 bp, Artificial Sequence
  primer
ccgcgtaaatcaccttgcggattcaggagactg

SEQ ID NO: 6 - DNA, 41 bp, Artificial Sequence
  primer
cggtacccggggatccggcaagtggttaaacccgctcaaac

SEQ ID NO: 7 - DNA, 80 bp, Artificial Sequence
  primer
gaaggcgtaggtgatgtgaagcctttcttataacaaattgcgttccacggatggaagacttctagacgctcaagttagta

SEQ ID NO: 8 - DNA, 80 bp, Artificial Sequence
  primer
aggcgcaacatactacactatctttatggttaaagcttgtttcgattaacatcagcttaaggcaagtggttaaacccgct

SEQ ID NO: 9 - DNA, 64 bp, Artificial Sequence
  primer
ttcacacaaataattttgtttaactttaagaaggagatataatgagccagaaaaccctgtttac

SEQ ID NO: 10 - DNA, 30 bp, Artificial Sequence
  primer
aattatttcagaacgcgtagttaaagttag

SEQ ID NO: 11 - DNA, 35 bp, Artificial Sequence; identical to SEQ ID NO: 3
  primer

SEQ ID NO: 12 - DNA, 27 bp, Artificial Sequence
  primer
aattatttgtgtgaaattgttatccgc

SEQ ID NO: 13 - DNA, 64 bp, Artificial Sequence
  primer
cgttctgaaataattttgtttaactttaagaaggagatataatgaagaaggtttggcttaaccg

SEQ ID NO: 14 - DNA, 41 bp, Artificial Sequence
  primer
cggtacccggggatccaccggcgtctgacgactgacttaac

SEQ ID NO: 15 - DNA, 80 bp, Artificial Sequence
  primer
tcgcgacggcaacatttcgctaaagtcacgccccttcttcaccggcatggggattatttctctagacgctcaagttagta

SEQ ID NO: 16 - DNA, 80 bp, Artificial Sequence
  primer
gcagcagaaaaactggctcaggcgcaggcagatttaagccgctgctgagccatttttcaaaccggcgtctgacgactgac

SEQ ID NO: 17 - DNA, 1683 bp, Bacillus subtilis
  CDS (1)..(1680)
atgcagtctcaaaagccgtggcttgccgagtatcccaacgatatcccgcatgagcttccgttgccgaataaaaccctgcaatccatcctgacagactccgccgcacgattccctgataaaaccgcaatatcgttttatggaaaaaaactcacctttcatgacattctgacggatgctcttaaacttgcagcttttttgcagtgcaatggcctgcaaaaaggagacagagtggctgttatgctgcccaattgcccgcaaacagtgatttcttattatggcgtcttgtttgccggtggcatcgtggtgcagacgaatccgctttataccgagcatgagcttgaataccagcttagagatgctcaggtaagtgtaatcatcaccttagatttgcttttcccgaaggcaataaaaatgaaaacgttatcaatagtcgaccagattttgataaccagtgtaaaagactatttgccttttccgaaaaatattctttacccgctgacgcaaaaacaaaaagtgcatatcgactttgacaaaacagctaacattcacacgttcgcttcctgtatgaagcaggaaaaaactgaactcctgacaatcccgaagatcgatcctgagcatgatatcgctgtcctccagtataccggaggaacaaccggagctccaaaaggggtcatgctcacgcaccaaaatattttggcaaatactgaaatgtgcgccgcttggatgtatgacgtgaaagaaggcgctgaaaaagtgctgggcatcgttccattttttcatgtctacggactgacggcagtcatgaactattcgattaaactaggcttcgaaatgattcttcttcctaagtttgatccgctcgaaacacttaaaatcatcgacaaacacaaaccgacgctctttcctggcgcaccgacaatttacattggccttttgcatcaccctgaattgcagcattatgatctgtcatccattaaaagctgtcttagcggatcagccgcgctccccgtggaggtaaagcagaaatttgaaaaggtgactggtggaaagcttgtggaaggctatggtttgtctgaggcatccccagtgacgcacgccaactttatctggggaaaaaacaagccgggcagtatcggctgtccttggccgagtacggacgctgcgatctattctgaagagacgggtgagcttgccgctccgtatgagcatggagaaatcattgtaaaaggtccgcaagtcatgaaaggatactggaataaaccagaggaaaccgccgcagtgctgagagatggctggctgttcaccggggacatgggctatatggatgaagaaggctttttctatattgctgacaggaagaaggatatcatcattgccggcggttacaatatttacccgcgtgaggtagaagaagcgctttatgaacatgaagccatccaggaaattgttgtcgcaggcgttcccgattcctacaggggagaaacagtaaaagcatttgtcgtattaaagaaaggcgcaaaagccgatacagaagaactggacgcttttgcgagatcacgtctagctccctataaggtgcccaaagcctatgagttcagaaaggagctgccgaaaacggcggtgggaaaaattttaagaaggcgtttacttgaagaagaaaccgaaaatcatcatatcaaataa

SEQ ID NO: 18 - protein, 560 aa, Bacillus subtilis
MQSQKPWLAEYPNDIPHELPLPNKTLQSILTDSAARFPDKTAISFYGKKLTFHDILTDALKLAAFLQCNGLQKGDRVAVMLPNCPQTVISYYGVLFAGGIVVQTNPLYTEHELEYQLRDAQVSVIITLDLLFPKAIKMKTLSIVDQILITSVKDYLPFPKNILYPLTQKQKVHIDFDKTANIHTFASCMKQEKTELLTIPKIDPEHDIAVLQYTGGTTGAPKGVMLTHQNILANTEMCAAWMYDVKEGAEKVLGIVPFFHVYGLTAVMNYSIKLGFEMILLPKFDPLETLKIIDKHKPTLFPGAPTIYIGLLHHPELQHYDLSSIKSCLSGSAALPVEVKQKFEKVTGGKLVEGYGLSEASPVTHANFIWGKNKPGSIGCPWPSTDAAIYSEETGELAAPYEHGEIIVKGPQVMKGYWNKPEETAAVLRDGWLFTGDMGYMDEEGFFYIADRKKDIIIAGGYNIYPREVEEALYEHEAIQEIVVAGVPDSYRGETVKAFVVLKKGAKADTEELDAFARSRLAPYKVPKAYEFRKELPKTAVGKILRRRLLEEETENHHIK

SEQ ID NO: 19 - DNA, 64 bp, Artificial Sequence; identical to SEQ ID NO: 9
  primer

SEQ ID NO: 20 - DNA, 30 bp, Artificial Sequence; identical to SEQ ID NO: 10
  primer

SEQ ID NO: 21 - DNA, 35 bp, Artificial Sequence; identical to SEQ ID NO: 3
  primer

SEQ ID NO: 22 - DNA, 27 bp, Artificial Sequence
  primer
aattatttgtgtgaaattgttatccgc

SEQ ID NO: 23 - DNA, 63 bp, Artificial Sequence
  primer
cgttctgaaataattttgtttaactttaagaaggagatataatgcagtctcaaaagccgtggc

SEQ ID NO: 24 - DNA, 41 bp, Artificial Sequence
  primer
cggtacccggggatcccataggcgttctctttttttatttg

SEQ ID NO: 25 - DNA, 80 bp, Artificial Sequence; identical to SEQ ID NO: 15
  primer

SEQ ID NO: 26 - DNA, 80 bp, Artificial Sequence
  primer
gcagcagaaaaactggctcaggcgcaggcagatttaagccgctgctgagccatttttcaagcgttctctttttttatttg

(19) United States
(12) Patent Application Publication
(10) Pub. No.: US 2015/0218605 A1
(43) Pub. Date: Aug. 6, 2015

(54) Method for Producing L-Amino Acid

(71) Applicant: AJINOMOTO CO., INC., TOKYO (JP)

(72) Inventors: Yasushi Hoshino, Kanagawa (JP); Yukiko Miyagawa, Kanagawa (JP); Seizaburo Shiraga, Kanagawa (JP)

(73) Assignee: AJINOMOTO CO., INC., TOKYO (JP)

(21) Appl. No.: 14/687,025

(22) Filed: Apr. 15, 2015

Related U.S. Application Data
(63) Continuation of application No. PCT/JP2013/078373, filed on Oct. 18, 2013.

(30) Foreign Application Priority Data
Oct. 19, 2012 (JP) 2012-231582

(51) Int. Cl. C12P 13/08

(52) U.S. Cl. CPC C12P 13/08

(57) ABSTRACT

A method for producing an L-amino acid using a fatty acid as the carbon source is provided. An L-amino acid is produced by culturing a bacterium belonging to the family Enterobacteriaceae and having an L-amino acid-producing ability, which has been introduced with the lcfA gene, in a medium containing a fatty acid, and collecting the L-amino acid from the medium.

This application is a Continuation of, and claims priority under 35 U.S.C. §120 to, International Application No. PCT/JP2013/078373, filed Oct. 18, 2013, and claims priority therethrough under 35 U.S.C. §119 to Japanese Patent Application No. 2012-231582, filed Oct. 19, 2012, the entireties of which are incorporated by reference herein. Also, the Sequence Listing filed electronically herewith is hereby incorporated by reference (File name: 2015-04-15T_US-533_Seq_List; File size: 18 KB; Date recorded: Apr. 15, 2015).

### BACKGROUND OF THE INVENTION

1. Field of the Invention

The present invention relates to a method for producing an L-amino acid such as L-lysine using a bacterium. L-amino acids are used in many and varied products, such seasonings, food additives, feed additives, chemicals, and drugs.

2. Brief Description of the Related Art

L-Amino acids are industrially produced by fermentation using L-amino acid-producing bacteria such as Escherichia bacteria having an L-amino acid-producing ability. As such L-amino acid-producing bacteria, strains isolated from the nature and modified strains thereof are used. Examples of the method for producing L-lysine include, for example, the methods described in Japanese Patent Laid-open (KOKAI) No. 10-165180, Japanese Patent Laid-open (KOKAI) No. 11-192088, Japanese Patent Laid-open (KOKAI) No. 2000-253879, and Japanese Patent Laid-open (KOKAI) No. 2001-057896.

In the production of L-amino acids by fermentation, saccharides such as glucose, fructose, sucrose, blackstrap molasses, and starch hydrolysates are generally used as the carbon source.

Methods are also known for producing an L-amino acid by fermentation using a fatty acid as the carbon source. Examples of such methods include, for example, a method of using an L-amino acid-producing bacterium belonging to the family Enterobacteriaceae, which has a mutant rpsA gene (International Patent Publication WO2011/096554), a method of using an L-amino acid-producing bacterium belonging to the family Enterobacteriaceae, which has been modified so that the activity of the UspA protein is decreased (International Patent Publication No. WO2011/096555), and a method of using an L-amino acid-producing bacterium belonging to the family Enterobacteriaceae, which has been modified so that the fatty acid-assimilation ability is increased (Japanese Patent Laid-open (KOKAI) No. 2011-167071).

Fatty acids are assimilated via the assimilation pathway called β-oxidation (Clark, D. P. and Cronan, J. E. Jr., 1996, pp. 343-357, In F. D. Neidhardt (ed.), Escherichia coli and Salmonella Cellular and Molecular Biology/Second Edition, American Society for Microbiology Press, Washington, D.C.). The enzymes that catalyze the β-oxidation reactions are encoded by the fad regulon which includes fadL, fadD, fadE, fadB, and fadA, and the expression of the fad regulon is suppressed by a transcription factor encoded by fadR (Clark, D. P. and Cronan, J. E. Jr., 1996, pp. 343-357, In F. D. Neidhardt (ed.), Escherichia coli and Salmonella Cellular and Molecular Biology/Second Edition, American Society for Microbiology Press, Washington, D.C.). Therefore, for example, by attenuating the expression of the fadR gene, or enhancing the expression of the fadL fadE, fadD, fadB, and fadA genes, the ability of a bacterium to assimilate fatty acids can be enhanced (Japanese Patent Laid-open (KOKAI) No. 2011-167071).

The fadD gene encodes a protein that is responsible for generating a fatty acyl-CoA from a long chain fatty acid, and takes up it through the inner membrane (Dirusso, C. C. and Black P. N., 2004, J. Biol. Chem., 279:49563-49566; Schmelter, T. et al., 2004, J. Biol. Chem., 279:24163-24170). For example, it is known that, by enhancing the expression of the fadD gene in Escherichia coli, production of an L-amino acid such as L-lysine and L-threonine can be improved (Japanese Patent Laid-open (KOKAI) No. 2011-167071). Bacillus subtilis has the lcfA gene as a gene corresponding to the fadD gene (Matsuoka H. et al., 2007, J. Biol. Chem., Vol. 282, No. 8, pp. 5180-5194).

However, the protein encoded by the lcfA gene of Bacillus subtilis has an identity of only 39% to the protein encoded by the fadD gene of Escherichia coli, and the influence of introducing the lcfA gene into Enterobacteraceae bacteria on L-amino acid production using a fatty acid as a carbon source has not been previously reported.

### SUMMARY OF THE INVENTION

The present invention is based on the development of a novel technique for improving an L-amino acid-producing ability of a bacterium when using a fatty acid as the carbon source, and provides a method for producing an L-amino acid using a fatty acid as the carbon source.

The lcfA gene derived from Bacillus subtilis can be introduced into a bacterium, and as a result, an L-amino acid-producing ability of the bacterium when using a fatty acid as the carbon source can be improved.

It is an aspect of the present invention to provide a method for producing an L-amino acid comprising:

culturing a bacterium belonging to the family Enterobacteriaceae and having an L-amino acid-producing ability in a medium comprising a fatty acid; and

collecting the L-amino acid from the medium,

wherein the lcfA gene has been introduced into the bacterium, and

the lcfA gene is a DNA selected from the group consisting of:

(A) a DNA encoding a protein comprising the amino acid sequence of SEQ ID NO: 18;

(B) a DNA encoding a protein comprising the amino acid sequence of SEQ ID NO: 18 including substitution, deletion, insertion, or addition of one or several amino acid residues, and having an activity of generating a fatty acyl-CoA from a long chain fatty acid, and taking it up through the inner membrane;

(C) a DNA comprising the nucleotide sequence of SEQ ID NO: 17; and

(D) a DNA hybridizable under stringent conditions with a nucleotide sequence complementary to the nucleotide sequence of SEQ ID NO: 17 or with a probe that can be prepared from the nucleotide sequence, and encoding a protein having an activity of generating a fatty acyl-CoA from a long chain fatty acid, and taking it up through the inner membrane.

It is a further aspect of the present invention to provide the method as described above, wherein the bacterium has been further modified so that fatty acid-assimilating ability thereof is increased.

It is a further aspect of the present invention to provide the method as described above, wherein the fatty acid-assimilating ability is increased by a method selected from the group consisting of:

(a) attenuating expression of the fadR gene,

(b) enhancing the expression of a gene selected from the group consisting of fadL, fadE, fadD, fadB, fadA and combinations thereof,

(c) enhancing expression of the cyoABCDE operon, and

(d) combinations thereof.

It is a further aspect of the present invention to provide the method as described above, wherein the fatty acid is oleic acid.

It is a further aspect of the present invention to provide the method as described above, wherein the medium further contains a carbon source in addition to the fatty acid.

It is a further aspect of the present invention to provide the method as described above, wherein the carbon source in addition to the fatty acid is glucose.

It is a further aspect of the present invention to provide the method as described above, wherein the L-amino acid is L-lysine.

It is a further aspect of the present invention to provide the method as described above, wherein the bacterium is an Escherichia bacterium.

It is a further aspect of the present invention to provide the method as described above, wherein the bacterium is Escherichia coli.

### DESCRIPTION OF THE PREFERRED EMBODIMENTS

<1> Bacterium

The bacterium (henceforth also referred to as “the bacterium of the present invention”) is a bacterium belonging to the family Enterobacteriaceae and having an L-amino acid-producing ability, and into which the lcfA gene has been introduced. The bacterium has an ability to utilize a fatty acid as the carbon source.

<1-1> Bacterium having L-amino acid-producing ability

A “bacterium having an L-amino acid-producing ability” refers to a bacterium having an ability to produce and accumulate an objective L-amino acid in a medium containing a fatty acid or the bacterial cells to such a degree that the L-amino acid can be collected, when the bacterium is cultured in the medium. The bacterium having an L-amino acid-producing ability may be a bacterium that can accumulate an objective L-amino acid in a medium or the cells in an amount larger than that obtainable with a non-modified strain. Examples of the non-modified strain include wild-type strains and parent strains. The bacterium having an L-amino acid-producing ability may be a bacterium that can accumulate an objective L-amino acid in a medium or the cells in an amount of 0.5 g/L or more, or 1.0 g/L or more.

Examples of the L-amino acid include basic amino acids such as L-lysine, L-ornithine, L-arginine, L-histidine, and L-citrulline; aliphatic amino acids such as L-isoleucine, L-alanine, L-valine, L-leucine, and glycine; amino acids which are hydroxy-monoaminocarboxylic acids such as L-threonine and L-serine; cyclic amino acids such as L-proline; aromatic amino acids such as L-phenylalanine, L-tyrosine, and L-tryptophan; sulfur-containing amino acids such as L-cysteine, L-cystine, and L-methionine; acidic amino acids such as L-glutamic acid and L-aspartic acid; and amino acids having an amide group in the side chain such as L-glutamine and L-asparagine. The bacterium can have an ability to produce two or more kinds of amino acids.

The L-amino acid may be an L-amino acid in free form, a salt thereof, or a mixture of them. Examples of the salt include, for example, sulfates, hydrochlorides, carbonates, ammonium salts, sodium salts, and potassium salts.

The amino acid is an L-amino acid unless otherwise stated.

Examples of bacteria belonging to the family Enterobacteriaceae include bacteria belonging to the genus Escherichia, Enterobacter, Pantoea, Klebsiella, Serratia, Erwinia, Photorhabdus, Providencia, Salmonella, Morganella, or the like. Specifically, bacteria classified into the family Enterobacteriaceae according to the taxonomy used in the NCBI (National Center for Biotechnology Information) database (http://www.ncbi.nlm.nih.gov/Taxonomy/Browser/wwwtax.cgi?id=91347) can be used.

The Escherichia bacteria are not particularly limited, and examples thereof include those classified into the genus Escherichia according to the taxonomy known to those skilled in the field of microbiology. Examples of the Escherichia bacteria include, for example, those described in the work of Neidhardt et al. (Backmann B. J., 1996, Derivations and Genotypes of some mutant derivatives of Escherichia coli K-12, pp. 2460-2488, Table 1, In F. D. Neidhardt (ed.), Escherichia coli and Salmonella Cellular and Molecular Biology, Second Edition, American Society for Microbiology Press, Washington, D.C.). Examples of the Escherichia bacterium include, for example, Escherichia coli. Specific examples of Escherichia coli include, for example, Escherichia coli W3110 (ATCC 27325) and Escherichia coli MG1655 (ATCC 47076) derived from the prototype wild-type strain, K12 strain.

These strains are available from, for example, the American Type Culture Collection (Address: 12301 Parklawn Drive, Rockville, Md. 20852, P.O. Box 1549, Manassas, Va. 20108, United States of America). That is, registration numbers are given to the respective strains, and the strains can be ordered by using these registration numbers (refer to http://www.atcc.org/). The registration numbers of the strains are listed in the catalogue of the American Type Culture Collection.

The Enterobacter bacteria are not particularly limited, and examples thereof include those classified into the genus Enterobacter according to classification known to a person skilled in the art of microbiology. Examples of the Enterobacter bacteria include, for example, Enterobacter agglomerans and Enterobacter aerogenes. Specific examples of Enterobacter agglomerans include, for example, the Enterobacter agglomerans ATCC 12287 strain. Specific examples of Enterobacter aerogenes include, for example, the Enterobacter aerogenes ATCC 13048 strain, NBRC 12010 strain (Biotechnol Bioeng., 2007, Mar. 27; 98(2):340-348), and AJ110637 strain (FERM ABP-10955). Examples the Enterobacter bacteria also include, for example, the strains described in European Patent Application Laid-open (EP-A) No. 0952221.

The Pantoea bacteria are not particularly limited, and examples include those classified into the genus Pantoea according to classification known to a person skilled in the art of microbiology. Examples of the Pantoea bacteria include, for example, Pantoea ananatis, Pantoea stewartii, Pantoea agglomerans, and Pantoea citrea. Specific examples of Pantoea ananatis include, for example, the Pantoea ananatis AJ13355 strain (FERM BP-6614), AJ13356 strain (FERM BP-6615), AJ13601 strain (FERM BP-7207), SC17 strain (FERM BP-11091), and SC17(0) strain (VKPM B-9246). Some strains of Enterobacter agglomerans were recently reclassified into Pantoea agglomerans, Pantoea ananatis, Pantoea stewartii, or the like on the basis of nucleotide sequence analysis of 16S rRNA etc. (Int. J. Syst. Bacteriol., 43, 162-173 (1993)). In the present invention, the Pantoea bacteria include those reclassified into the genus Pantoea as described above.

Examples of the Erwinia bacteria include Erwinia amylovora and Erwinia carotovora. Examples of the Klebsiella bacteria include Klebsiella planticola.

An L-amino acid-producing bacterium belonging to the family Enterobacteriaceae can be obtained by imparting an L-amino acid-producing ability to such a bacterium belonging to the family Enterobacteriaceae as mentioned above, or enhancing an L-amino acid-producing ability of such a bacterium belonging to the family Enterobacteriaceae as mentioned above.

An L-amino acid-producing ability can be imparted or enhanced by methods conventionally employed in the breeding of amino acid-producing strains of coryneform bacteria, Escherichia bacteria, and so forth (see “Amino Acid Fermentation”, Gakkai Shuppan Center (Ltd.), 1st Edition, published May 30, 1986, pp. 77-100). Examples of such methods include, for example, acquiring an auxotrophic mutant strain, acquiring an L-amino acid analogue-resistant strain, acquiring a metabolic regulation mutant strain, and constructing a recombinant strain in which the activity of an L-amino acid biosynthetic enzyme is enhanced. In the breeding of L-amino acid-producing bacteria, one of the above-described properties such as auxotrophy, analogue resistance, and metabolic regulation mutation may be imparted alone, or two or three or more of such properties may be imparted in combination. Also, in the breeding of an L-amino acid-producing bacterium, the activity of one of L-amino acid biosynthetic enzymes may be enhanced alone, or the activities of two or three or more of such enzymes may be enhanced in combination. Furthermore, imparting property(s) such as auxotrophy, analogue resistance, and metabolic regulation mutation can be combined with enhancing the activity(s) of biosynthetic enzyme(s).

An auxotrophic mutant strain, L-amino acid analogue-resistant strain, or metabolic regulation mutant strain having an L-amino acid-producing ability can be obtained by subjecting a parent strain or wild-type strain to a usual mutagenesis treatment, and then selecting a strain exhibiting autotrophy, analogue resistance, or metabolic regulation mutation, and having an L-amino acid-producing ability from the obtained mutant strains. Examples of the usual mutagenesis treatment include irradiation of X-ray or ultraviolet and a treatment with a mutation agent such as N-methyl-N′-nitro-N-nitrosoguanidine.

An L-amino acid-producing ability can also be imparted or enhanced by enhancing the activity of an enzyme involved in biosynthesis of an objective L-amino acid. An enzyme activity can be enhanced by, for example, modifying a bacterium so that the expression of a gene encoding the enzyme is enhanced. Methods for enhancing gene expression are described in WO00/18935, EP 1010755 A, and so forth. The detailed procedures for enhancing enzyme activity will be described later.

Further, an L-amino acid-producing ability can also be imparted or enhanced by reducing the activity of an enzyme that catalyzes a reaction branching away from the biosynthetic pathway of an objective L-amino acid to generate a compound other than the objective L-amino acid. The “enzyme that catalyzes a reaction branching away from the biosynthetic pathway of an objective L-amino acid to generate a compound other than the objective L-amino acid” includes an enzyme involved in decomposition of the objective amino acid. The methods for reducing an enzyme activity will be described later.

Hereafter, L-amino acid-producing bacteria and methods for imparting or enhancing an L-amino acid-producing ability will be specifically exemplified. All of the properties of the L-amino acid-producing bacteria and modifications for imparting or enhancing an L-amino acid-producing ability exemplified below may be used independently or in any appropriate combination.

<L-Lysine-Producing Bacteria>

Examples of L-lysine-producing bacteria and parent strains that can be used to derive them include strains in which the activity or activities of one or more kinds of L-lysine biosynthetic enzymes have been enhanced. Examples of such enzymes include, but are not particularly limited to, dihydrodipicolinate synthase (dapA), aspartokinase III (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), aspartate aminotransferase (aspartate transaminase) (aspC), diaminopimelate epimerase (dapF), tetrahydrodipicolinate succinylase (dapD), succinyl diaminopimelate deacylase (dapE), and aspartase (aspA) (EP 1253195 A). Abbreviations of the gene names are indicated in the parentheses (the same shall apply to the following descriptions). Among these enzymes, the activity or activities of one or more kinds of enzymes such as dihydrodipicolinate reductase, diaminopimelate decarboxylase, diaminopimelate dehydrogenase, phosphoenolpyrvate carboxylase, aspartate aminotransferase, diaminopimelate epimerase, aspartate semialdehyde dehydrogenease, tetrahydrodipicolinate succinylase, and succinyl diaminopimelate deacylase can be enhanced. In addition, L-lysine-producing bacteria and parent strains that can be used to derive them can express an increased level of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), or combinations of these. Since aspartokinase III (lysC) is subject to feedback inhibition by L-lysine, a mutant lysC gene encoding an aspartokinase III desensitized to feedback inhibition by L-lysine (U.S. Pat. No. 5,932,453) may be used for enhancing the activity of this enzyme. Further, since dihydrodipicolinate synthase (dapA) is subject to feedback inhibition by L-lysine, a mutant dapA gene encoding a dihydrodipicolinate synthase desensitized to feedback inhibition by L-lysine may be used for enhancing the activity of this enzyme.

Examples of L-lysine-producing bacteria and parent strains that can be used to derive them also include strains in which the activity or activities of one or more kinds of enzymes that catalyze a reaction branching away from the biosynthetic pathway of L-lysine to generate a compound other than L-lysine have been reduced or eliminated. Examples of such enzymes include, but are not particularly limited to, homoserine dehydrogenase, lysine decarboxylase (U.S. Pat. No. 5,827,698), and malic enzyme (WO2005/010175).

Examples of L-lysine-producing bacteria and parent strains that can be used to derive them also include mutants having resistance to an L-lysine analogue. L-Lysine analogues inhibit the growth of bacteria belonging to the family Enterobacteriaceae such as Escherichia bacteria, but this inhibition is fully or partially released when L-lysine is present in the medium. Examples of the L-lysine analogues include, but are not particularly limited to, oxalysine, lysine hydroxamate, S-(2-aminoethyl)-L-cysteine (AEC), γ-methyllysine, and α-chlorocaprolactam. Mutant strains having resistance to these lysine analogues can be obtained by subjecting a bacterium belonging to the family Enterobacteriaceae to a conventional artificial mutagenesis treatment.

Specific examples of L-lysine-producing bacteria and parent strains that can be used to derive them include, but are not particularly limited to, E. coli AJ11442 (FERM BP-1543, NRRL B-12185, see U.S. Pat. No. 4,346,170) and E. coli VL611. In these strains, aspartokinase is desensitized to feedback inhibition by L-lysine.

Specific examples of L-lysine-producing bacteria and parent strains that can be used to derive them also include the E. coli WC196 strain. The WC196 strain was bred by imparting AEC resistance to the W3110 strain, which was derived from E. coli K-12 (U.S. Pat. No. 5,827,698). The WC196 strain was designated as E. coli AJ13069 and deposited at the National Institute of Bioscience and Human-Technology, Agency of Industrial Science and Technology (currently, independent administrative agency, National Institute of Technology and Evaluation, International Patent Organism Depositary, #120, 2-5-8 Kazusakamatari, Kisarazu-shi, Chiba-ken, 292-0818, Japan) on Dec. 6, 1994 and assigned an accession number of FERM P-14690. Then, the deposit was converted to an international deposit under the provisions of the Budapest Treaty on Sep. 29, 1995, and assigned an accession number of FERM BP-5252 (U.S. Pat. No. 5,827,698).

Examples of L-lysine-producing bacteria include E. coli WC196ΔcadAΔldc and E. coli WC196ΔcadAΔldc/pCABD2 (WO2006/078039). The E. coli WC196ΔcadAΔldc is a strain constructed from the WC196 strain by disrupting the cadA and ldcC genes encoding lysine decarboxylase. The WC196ΔcadAΔldc/pCABD2 strain was obtained by introducing the plasmid pCABD2 containing lysine biosynthesis enzyme genes (U.S. Pat. No. 6,040,160) into the WC196ΔcadAΔldc strain. The WC196ΔcadAΔldc strain, designated as AJ110692, was deposited at the independent administrative agency, National Institute of Advanced Industrial Science and Technology, International Patent Organism Depositary (currently, independent administrative agency, National Institute of Technology and Evaluation, International Patent Organism Depositary, #120, 2-5-8 Kazusakamatari, Kisarazu-shi, Chiba-ken, 292-0818, Japan) on Oct. 7, 2008, and assigned an accession number of FERM BP-11027. The plasmid pCABD2 contains a mutant dapA gene derived from Escherichia coli and encoding a dihydrodipicolinate synthase (DDPS) having a mutation for desensitization to feedback inhibition by L-lysine, a mutant lysC gene derived from Escherichia coli and encoding aspartokinase III having a mutation for desensitization to feedback inhibition by L-lysine, the dapB gene derived from Escherichia coli and encoding dihydrodipicolinate reductase, and the ddh gene derived from Brevibacterium lactofermentum and encoding diaminopimelate dehydrogenase.

<L-Threonine-Producing Bacteria>

Examples of L-threonine-producing bacteria and parent strains that can be used to derive them include strains in which the activity or activities of one or more L-threonine biosynthetic enzymes have been enhanced. Examples of such enzymes include, but are not particularly limited to, aspartokinase III (lysC), aspartate semialdehyde dehydrogenase (asd), aspartokinase I (thrA), homoserine kinase (thrB), threonine synthase (thrC), and aspartate aminotransferase (aspartate transaminase) (aspC). Among these enzymes, activity or activities of one or more kinds of enzymes such as aspartokinase III, aspartate semialdehyde dehydrogenase, aspartokinase I, homoserine kinase, aspartate aminotransferase, and threonine synthase can be enhanced. The genes encoding the L-threonine biosynthesis enzymes can be introduced into a bacterium having a reduced ability to decompose threonine. Examples of such a strain in which threonine decomposition is suppressed include, for example, the E. coli TDH6 strain, which is deficient in the threonine dehydrogenase activity (Japanese Patent Laid-open (Kokai) No. 2001-346578).

The activities of the L-threonine biosynthesis enzymes are inhibited by the endproduct, L-threonine. Therefore, for constructing L-threonine-producing strains, the genes of the L-threonine biosynthesis enzymes can be modified so that the enzymes are desensitized to feedback inhibition by L-threonine. The aforementioned thrA, thrB, and thrC genes constitute the threonine operon, which forms an attenuator structure. The expression of the threonine operon is inhibited by isoleucine and threonine in the culture medium and also suppressed by attenuation. Therefore, expression of the threonine operon can be enhanced by removing the leader sequence or the attenuator in the attenuation region (refer to Lynn, S. P., Burton, W. S., Donohue, T. J., Gould, R. M., Gumport, R. I, and Gardner, J. F., J. Mol. Biol. 194:59-69 (1987); WO02/26993; WO2005/049808; and WO2003/097839).

The native promoter of the threonine operon is present upstream of the threonine operon, and can be replaced with a non-native promoter (refer to WO98/04715). Also, the threonine operon may be constructed so that the expression of the threonine biosynthesis genes is controlled by the repressor and promoter of λ-phage (European Patent No. 0593792). Furthermore, a bacterium modified so as to be desensitized to feedback inhibition by L-threonine can also be obtained by selecting a strain resistant to α-amino-β-hydroxyisovaleric acid (AHV), which is an L-threonine analogue.

The expression amount of the threonine operon that has been modified so as to be desensitized to feedback inhibition by L-threonine as described above can be increased in a host by increasing the copy number thereof or by ligating it to a potent promoter. The copy number can be increased by introducing a plasmid carrying the threonine operon into a host. The copy number can also be increased by transferring the threonine operon to the genome of a host using a transposon, Mu-phage, or the like.

Examples of methods for imparting or enhancing L-threonine-producing ability also include, for example, a method of imparting L-threonine resistance to a host, and a method of imparting L-homoserine resistance to a host. Such resistance can be imparted by, for example, enhancing expression of a gene that imparts L-threonine resistance or a gene that imparts L-homoserine resistance. Examples of the genes that impart the above-mentioned resistance include the rhtA gene (Res. Microbiol. 154:123-135 (2003)), rhtB gene (EP 0994190 A), rhtC gene (EP 1013765 A), yfiK gene, and yeaS gene (EP 1016710 A). As for methods for imparting L-threonine resistance to a host, those described in EP 0994190 A and WO90/04636 can be referred to.

Specific examples of L-threonine-producing bacteria and parent strains that can be used to derive them include, but are not particularly limited to, strains belonging to the genus Escherichia, such as E. coli TDH-6/pVIC40 (VKPM B-3996, U.S. Pat. Nos. 5,175,107 and 5,705,371), E. coli 472T23/pYN7 (ATCC 98081, U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A), and E. coli VKPM B-5318 (EP 0593792 B).

The VKPM B-3996 strain is a strain obtained by introducing the plasmid pVIC40 into the TDH-6 strain. The TDH-6 strain has sucrose-assimilating ability and is deficient in the thrC gene, and the ilvA gene thereof has a leaky mutation. This VKPM B-3996 strain also has a mutation in the rhtA gene, which imparts resistance to high concentration of threonine or homoserine. The plasmid pVIC40 is a plasmid obtained by inserting the thrA*BC operon containing a mutant thrA gene encoding an aspartokinase-homoserine dehydrogenase I resistant to feedback inhibition by threonine and wild-type thrBC genes into an RSF1010-derived vector (U.S. Pat. No. 5,705,371). This mutant thrA gene encodes an aspartokinase-homoserine dehydrogenase I which is substantially desensitized to feedback inhibition by threonine. The B-3996 strain was deposited on Nov. 19, 1987 at the All-Union Scientific Center of Antibiotics (Nagatinskaya Street 3-A, 117105 Moscow, Russia) under the accession number RIA 1867. This strain was also deposited at the Russian National Collection of Industrial Microorganisms (VKPM, 1 Dorozhny proezd., 1 Moscow 117545, Russia) on Apr. 7, 1987 under the accession number B-3996.

The VKPM B-5318 strain is prototrophic with regard to isoleucine, and harbors the plasmid pPRT614, which corresponds to the plasmid pVIC40 of which the regulatory region of the threonine operon is replaced with a temperature-sensitive λ-phage Cl repressor and PR promoter. The VKPM B-5318 strain was deposited at the Russian National Collection of Industrial Microorganisms (VKPM, 1 Dorozhny proezd., 1 Moscow 117545, Russia) on May 3, 1990 under the accession number of VKPM B-5318.

The thrA gene encoding aspartokinase-homoserine dehydrogenase I of E. coli has been elucidated (nucleotide numbers 337 to 2799, GenBank accession NC_{—}000913.2, gi: 49175990). The thrA gene is located between the thrL and thrB genes on the chromosome of E. coli K-12. The thrB gene encoding homoserine kinase of Escherichia coli has been elucidated (nucleotide numbers 2801 to 3733, GenBank accession NC_{—}000913.2, gi: 49175990). The thrB gene is located between the thrA and thrC genes on the chromosome of E. coli K-12. The thrC gene encoding threonine synthase of E. coli has been elucidated (nucleotide numbers 3734 to 5020, GenBank accession NC_{—}000913.2, gi: 49175990). The thrC gene is located between the thrB gene and the yaaX open reading frame on the chromosome of E. coli K-12. The thrA*BC operon containing a mutant thrA gene encoding aspartokinase-homoserine dehydrogenase I resistant to feedback inhibition by threonine and the wild-type thrBC genes can be obtained from the well-known plasmid pVIC40, which is present in the threonine-producing strain E. coli VKPM B-3996 (U.S. Pat. No. 5,705,371).

The rhtA gene of E. coli is located at 18 min on the E. coli chromosome close to the g1nHPQ operon, which encodes components of the glutamine transport system. The rhtA gene is identical to ORF1 (ybiF gene, nucleotide numbers 764 to 1651, GenBank accession number AAA218541, gi:440181) and locates between the pexB and ompX genes. The unit that expresses a protein encoded by the ORF1 has been designated rhtA gene (rht: resistance to homoserine and threonine). It has also been revealed that the rhtA23 mutation that imparts resistance to high concentration of threonine or homoserine is an A-for-G substitution at position −1 with respect to the ATG start codon (ABSTRACTS of the 17th International Congress of Biochemistry and Molecular Biology in conjugation with Annual Meeting of the American Society for Biochemistry and Molecular Biology, San Francisco, Calif., Aug. 24-29, 1997, abstract No. 457; EP 1013765 A).

The asd gene of E. coli has already been elucidated (nucleotide numbers 3572511 to 3571408, GenBank accession NC_{—}000913.1, gi:16131307), and can be obtained by PCR (refer to White, T. J., et al., Trends Genet., 5, 185-189, 1989) utilizing primers prepared on the basis of the nucleotide sequence of the gene. The asd genes of other microorganisms can also be obtained in a similar manner.

The aspC gene of E. coli has also already been elucidated (nucleotide numbers 983742 to 984932, GenBank accession NC_{—}000913.1, gi:16128895), and can be obtained by PCR utilizing primers prepared on the basis of the nucleotide sequence of the gene. The aspC genes of other microorganisms can also be obtained in a similar manner.

<L-Arginine-Producing Bacteria>

Examples of L-arginine-producing bacteria and parent strains that can be used to derive them include strains in which the activity or activities of one or more L-arginine biosynthetic enzymes have been enhanced. Examples of such enzymes include, but are not particularly limited to, N-acetylglutamylphosphate reductase (argC), ornithine acetyl transferase (argJ), N-acetylglutamate kinase (argB), acetylornithine transaminase (argD), ornithine carbamoyl transferase (argF), argininosuccinate synthetase (argG), argininosuccinate lyase (argH), and carbamoyl phosphate synthetase (carAB). As the N-acetylglutamate synthase gene (argA), for example, a gene encoding a mutant N-acetylglutamate synthase desensitized to feedback inhibition by L-arginine by substitution for the amino acid residues corresponding to positions 15 to 19 of the wild type enzyme (EP 1170361 A) is a particular example.

Specific examples of L-arginine-producing bacteria and parent strains that can be used to derive them include, but are not particularly limited, strains belonging to the genus Escherichia, such as the E. coli 237 strain (VKPM B-7925) (U.S. Patent Published Application No. 2002/058315A1), derivative strains thereof harboring a mutant N-acetyl glutamate synthase (Russian Patent Application No. 2001112869), E. coli 382 strain derived from the 237 strain and having an improved acetic acid-assimilating ability (VKPM B-7926, EP 1170358 A1), and E. coli L-arginine-producing strain introduced with the argA gene encoding N-acetyl glutamate synthetase (EP 1170361 A1). The E. coli 237 strain was deposited at the Russian National Collection of Industrial Microorganisms (VKPM, 1 Dorozhny proezd., 1 Moscow 117545, Russia) on Apr. 10, 2000 under an accession number of VKPM B-7925, and the original deposit was converted to an international deposit under the provisions of the Budapest Treaty on May 18, 2001. The E. coli 382 strain was deposited at the Russian National Collection of Industrial Microorganisms (VKPM, 1 Dorozhny proezd., 1 Moscow 117545, Russia) on Apr. 10, 2000 under accession number of VKPM B-7926.

Examples of L-arginine-producing bacteria and parent strains that can be used to derive them also include strains having resistance to amino acid analogues, and so forth. Examples of such strains include Escherichia coli mutant strains having resistance to α-methylmethionine, p-fluorophenylalanine, D-arginine, arginine hydroxamate, S-(2-aminoethyl)-cysteine, α-methylserine, β-2-thienylalanine, or sulfaguanidine (refer to Japanese Patent Laid-open (KOKAI) No. 56-106598).

<L-Citrulline-Producing Bacteria and L-Ornithine-Producing Bacteria>

The biosynthetic pathways of L-citrulline and L-ornithine are common to that of L-arginine. Therefore, an ability to produce L-citrulline and/or L-ornithine can be imparted or enhanced by increasing the activity or activities of N-acetylglutamate synthase (argA), N-acetylglutamylphosphate reductase (argC), ornithine acetyltransferase (argJ), N-acetylglutamate kinase (argB), acetylornithine transaminase (argD), and/or acetylornithine deacetylase (argE) (WO2006/35831).

<L-Histidine-Producing Bacteria>

Examples of L-histidine-producing bacteria and parent strains that can be used to derive them include strains in which the activity or activities of one or more L-histidine biosynthetic enzymes have been enhanced. Examples of such enzymes include, but are not particularly limited to, ATP phosphoribosyltransferase (hisG), phosphoribosyl AMP cyclohydrolase (hist), phosphoribosyl-ATP pyrophosphohydrolase (hisI), phosphoribosylformimino-5-aminoimidazole carboxamide ribotide isomerase (hisA), amidotransferase (hisH), histidinol phosphate aminotransferase (hisC), histidinol phosphatase (hisB), and histidinol dehydrogenase (hisD).

It is known that the L-histidine biosynthesis enzymes encoded by hisG and hisBHAFI are inhibited by L-histidine. Therefore, the ability to produce L-histidine can be imparted or enhanced by, for example, introducing a mutation for conferring resistance to feedback inhibition into the gene encoding ATP phosphoribosyltransferase (hisG) (Russian Patent Nos. 2003677 and 2119536).

Specific examples of L-histidine-producing bacteria and parent strains that can be used to derive them include, but are not particularly limited to, bacteria belonging to the genus Escherichia, such as the E. coli strain 24 (VKPM B-5945, RU 2003677), E. coli strain 80 (VKPM B-7270, RU 2119536), E. coli NRRL B-12116 to B12121 (U.S. Pat. No. 4,388,405), E. coli H-9342 (FERM BP-6675) and H-9343 (FERM BP-6676) (U.S. Pat. No. 6,344,347), E. coli H-9341 (FERM BP-6674) (EP 1085087), E. coli AI80/pFM201 (U.S. Pat. No. 6,258,554), E. coli FERM-P 5038 and 5048, which have been introduced with a vector carrying a DNA encoding an L-histidine-biosynthesis enzyme (Japanese Patent Laid-open (KOKAI) No. 56-005099), E. coli strains introduced with a gene for amino acid transport (EP 1016710 A), and E. coli strain 80 imparted with sulfaguanidine, DL-1,2,4-triazole-3-alanine, and streptomycin resistance (VKPM B-7270, Russian Patent No. 2119536).

<L-Cysteine-Producing Bacteria>

Examples of methods for imparting or enhancing L-cysteine-producing ability include, for example, a method of modifying a bacterium so that the bacterium has an increased activity or activities of one or more L-cysteine biosynthesis enzymes. Examples of such enzymes include, but are not particularly limited to, serine acetyltransferase and 3-phosphoglycerate dehydrogenase. The serine acetyltransferase activity can be enhanced by, for example, introducing a mutant cysE gene encoding a mutant serine acetyltransferase resistant to feedback inhibition by cysteine into a bacterium. Such a mutant serine acetyltransferase is disclosed in, for example, Japanese Patent Laid-open (KOKAI) No. 11-155571 and U.S. Patent Published Application No. 20050112731. Further, the 3-phosphoglycerate dehydrogenase activity can be enhanced by, for example, introducing a mutant serA gene encoding a mutant 3-phosphoglycerate dehydrogenase resistant to feedback inhibition by serine into a bacterium. Such a mutant 3-phosphoglycerate dehydrogenase is disclosed in, for example, U.S. Pat. No. 6,180,373.

Further, examples of methods for imparting or enhancing L-cysteine-producing ability also include, for example, a method of modifying a bacterium so that the bacterium has a reduced activity or activities of one or more enzymes that catalyze a reaction branching away from the biosynthesis pathway of L-cysteine to generate a compound other than L-cysteine. Examples of such enzymes include, for example, enzymes involved in decomposition of L-cysteine. Examples of the enzymes involved in decomposition of L-cysteine include, but are not particularly limited to, cystathionine-β-lyase (metC, Japanese Patent Laid-open (KOKAI) No. 11-155571; Chandra et al., Biochemistry, 21 (1982) 3064-3069), tryptophanase (tnaA, Japanese Patent Laid-open (KOKAI) No. 2003-169668; Austin Newton et al., J. Biol. Chem., 240 (1965) 1211-1218), O-acetylserine sulfhydrylase B (cysM, Japanese Patent Laid-open (KOKAI) No. 2005-245311), the malY gene product (Japanese Patent Laid-open (KOKAI) No. 2005-245311), and the d0191 gene product of Pantoea ananatis (Japanese Patent Laid-open (KOKAI) No. 2009-232844).

Further, examples of methods for imparting or enhancing L-cysteine-producing ability also include, for example, a method of enhancing the L-cysteine excretory system, and a method of enhancing the sulfate/thiosulfate transport system. Examples of proteins of the L-cysteine excretory system include the protein encoded by the ydeD gene (Japanese Patent Laid-open (KOKAI) No. 2002-233384), the protein encoded by the yfiK gene (Japanese Patent Laid-open (KOKAI) No. 2004-49237), the proteins encoded by the emrAB, emrKY, yojlH, acrEF, bcr, and cusA genes (Japanese Patent Laid-open (KOKAI) No. 2005-287333), and the protein encoded by the yeaS gene (Japanese Patent Laid-open (KOKAI) No. 2010-187552). Examples of the proteins of the sulfate/thiosulfate transport system include the proteins encoded by the cysPTWAM gene cluster.

Specific examples of L-cysteine-producing bacteria and parent strains that can be used to derive them include, but are not particularly limited to, strains belonging to the genus Escherichia, such as E. coli JM15 transformed with different cysE alleles encoding feedback-resistant serine acetyltransferases (U.S. Pat. No. 6,218,168, Russian patent application 2003121601), E. coli W3110 having over-expressed genes encoding proteins suitable for secreting cytotoxic substances (U.S. Pat. No. 5,972,663), E. coli strains having a reduced cysteine desulfohydrase activity (JP 11155571 A2), and E. coli W3110 having increased activity of a positive transcriptional regulator for cysteine regulon encoded by the cysB gene (WO0127307A1).

<L-Methionine-Producing Bacteria>

Specific examples of L-methionine-producing bacteria and parent strains that can be used to derive them include, but are not particularly limited to, L-threonine auxotrophic strains and mutant strains resistant to norleucine (Japanese Patent Laid-open (KOKAI) No. 2000-139471). Examples of L-methionine-producing bacteria and parent strains that can be used to derive them also include a strain containing a mutant homoserine transsuccinylase resistant to feedback inhibition by L-methionine (Japanese Patent Laid-open (KOKAI) No. 2000-139471, U.S. Patent Published Application No. 20090029424). Since L-methionine is biosynthesized via L-cysteine as an intermediate, L-methionine-producing ability can also be improved by improving L-cysteine-producing ability (Japanese Patent Laid-open (KOKAI) No. 2000-139471, U.S. Patent Published Application No. 20080311632).

Specific examples of L-methionine-producing bacteria and parent strains that can be used to derive them include, for example, E. coli AJ11539 (NRRL B-12399), E. coli AJ11540 (NRRL B-12400), E. coli AJ11541 (NRRL B-12401), E. coli AJ11542 (NRRL B-12402, British Patent No. 2075055), the E. coli 218 strain (VKPM B-8125, Russian Patent No. 2209248) and the 73 strain (VKPM B-8126, Russian Patent No. 2215782), which are resistant to norleucine, which is an analogue of L-methionine, and E. coli AJ13425 (FERMP-16808, Japanese Patent Laid-open (Kokai) No. 2000-139471). The AJ13425 strain is an L-threonine auxotrophic strain derived from the E. coli W3110, in which the methionine repressor is deleted, the intracellular S-adenosylmethionine synthetase activity is attenuated, and the intracellular homoserine transsuccinylase activity, cystathionine y-synthase activity, and aspartokinase-homoserine dehydrogenase II activity are enhanced.

<L-Leucine-Producing Bacteria>

Examples of L-leucine-producing bacteria and parent strains that can be used to derive them include strains in which the activity or activities of one or more L-leucine biosynthesis enzymes have been enhanced. Examples of such enzymes include, but are not particularly limited to, the enzymes encoded by the genes of the leuABCD operon. Further, for enhancing the activity of such an enzyme, for example, the mutant leuA gene encoding an isopropyl maleate synthase desensitized to feedback inhibition by L-leucine (U.S. Pat. No. 6,403,342) can be used.

Specific examples of L-leucine-producing bacteria and parent strains that can be used to derive them include, but are not particularly limited to, strains belonging to the genus Escherichia, such as E. coli strains resistant to leucine (for example, the 57 strain (VKPM B-7386, U.S. Pat. No. 6,124,121)), E. coli strains resistant to a leucine analogue such as β-2-thienylalanine, 3-hydroxyleucine, 4-azaleucine, and 5,5,5-trifluoroleucine (Japanese Patent Publication (KOKOKU) No. 62-34397 and Japanese Patent Laid-open (KOKAI) No. 8-70879), E. coli strains obtained by a gene engineering technique described in WO96/06926, and E. coli H-9068 (Japanese Patent Laid-open (KOKAI) No. 8-70879).

<L-Isoleucine-Producing Bacteria>

Examples of methods for imparting or enhancing L-isoleucine-producing ability include, for example, a method of modifying a bacterium so that the bacterium has an increased activity or activities of one or more L-isoleucine biosynthesis enzymes. Examples of such enzymes include, but are not particularly limited to, threonine deaminase and acetohydroxy acid synthase (Japanese Patent Laid-open (KOKAI) No. 2-458, FR 0356739, U.S. Pat. No. 5,998,178).

Examples of L-isoleucine-producing bacteria and parent strains that can be used to derive them include, but are not limited to, mutant strains having resistance to 6-dimethylaminopurine (Japanese Patent Laid-open (KOKAI) No. 5-304969), mutant strains having resistance to an isoleucine analogue such as thiaisoleucine and isoleucine hydroxamate, and mutant strains further having resistance to DL-ethionine and/or arginine hydroxamate (Japanese Patent Laid-open (KOKAI) No. 5-130882).

<L-Valine-Producing Bacteria>

Examples of L-valine-producing bacteria and parent strains that can be used to derive them include strains in which the activity or activities of one or more L-valine biosynthesis enzymes have been increased. Examples of such enzymes include, but are not particularly limited to, the enzymes encoded by the genes of the ilvGMEDA operon and the enzymes encoded by the genes of the ilvBNC operon. The ilvBN gene encodes acetohydroxy acid synthase, and the ilvC gene encodes isomero-reductase (WO00/50624). Expressions of the ilvGMEDA operon and the ilvBNC operon are suppressed (attenuated) by L-valine, L-isoleucine, and/or L-leucine. Therefore, for enhancing the activity of such an enzyme, it is preferred that the suppression of expression by the produced L-valine is released by removing or modifying a region required for the attenuation. Further, the threonine deaminase encoded by the ilvA gene is an enzyme that catalyzes the deamination reaction of L-threonine resulting 2-ketobutyric acid, which is the rate-limiting step of the L-isoleucine biosynthesis system. Therefore, for L-valine production, the ilvA gene can be, for example, disrupted, and the threonine deaminase activity is thereby decreased.

Examples of L-valine-producing bacteria and parent strains that can be used to derive them also include strains in which the activity or activities of one or more enzymes that catalyze a reaction branching away from the biosynthesis pathway of L-valine to generate a compound other than L-valine have been reduced. Examples of such enzymes include, but not particularly limited to, threonine dehydratase involved in the L-leucine synthesis, and the enzymes involved in the D-pantothenic acid synthesis (WO00/50624).

Specific examples of L-valine-producing bacteria and parent strains that can be used to derive them include, but are not particularly limited to, strains belonging to the genus Escherichia, such as E. coli strains modified so as to overexpress the ilvGMEDA operon (U.S. Pat. No. 5,998,178).

Examples of L-valine-producing bacteria and parent strains that can be used to derive them also include mutant strains having a mutation in amino-acyl t-RNA synthetase (U.S. Pat. No. 5,658,766). Examples of such strains include, for example, E. coli VL1970, which has a mutation in the ileS gene encoding isoleucine t-RNA synthetase. E. coli VL1970 was deposited at the Russian National Collection of Industrial Microorganisms (VKPM, 1 Dorozhny Proezd, 1 Moscow 117545, Russia) on Jun. 24, 1988 under the accession number of VKPM B-4411. Examples of L-valine-producing bacteria and parent strains that can be used to derive them also include mutant strains requiring lipoic acid for growth and/or lacking H⁺-ATPase (WO96/06926).

<L-Glutamic Acid-Producing Bacteria>

Examples of L-glutamic acid-producing bacteria and parent strains that can be used to derive them include strains in which the activity or activities of one or more L-glutamic acid biosynthetic enzymes have been enhanced. Examples of such enzymes include, but are not particularly limited to, glutamate dehydrogenase (gdhA), glutamine synthetase (glnA), glutamate synthetase (gltBD), isocitrate dehydrogenase (icdA), aconitate hydratase (acnA, acnB), citrate synthase (gltA), methylcitrate synthase (prpC), phosphoenolpyruvate carboxylase (ppc), pyruvate dehydrogenase (aceEF, lpdA), pyruvate kinase (pykA, pykF), phosphoenolpyruvate synthase (ppsA), enolase (eno), phosphoglyceromutase (pgmA, pgmI), phosphoglycerate kinase (pgk), glyceraldehyde-3-phophate dehydrogenase (gapA), triose phosphate isomerase (tpiA), fructose bisphosphate aldolase (fbp), phosphofructokinase (pfkA, pfkB), glucose phosphate isomerase (pgi), 6-phosphogluconate dehydratase (edd), 2-keto-3-deoxy-6-phosphogluconate aldolase (eda), and transhydrogenase. Among these enzymes, the activity or activities of one or more of glutamate dehydrogenase, citrate synthase, phosphoenol pyruvate carboxylase, and methylcitrate synthase can be enhanced.

Examples of strains belonging to the family Enterobacteriaceae and modified so that expression of the citrate synthase gene, phosphoenolpyruvate carboxylase gene, and/or glutamate dehydrogenase gene is increased include those disclosed in EP 1078989 A, EP 955368 A, and EP 952221 A. Further, examples of strains belonging to the family Enterobacteriaceae and modified so that the expression of a gene of the Entner-Doudoroff pathway (edd, eda) is increased include those disclosed in EP 1352966 B.

Further, examples of L-glutamic acid-producing bacteria and parent strains that can be used to derive them also include strains in which the activity or activities of one or more enzymes that catalyze a reaction branching away from the L-glutamic acid biosynthesis pathway to generate a compound other than L-glutamic acid have been decreased or eliminated. Examples of such enzymes include, but are not particularly limited to, isocitrate lyase (aceA), α-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (ldh), glutamate decarboxylase (gadAB), succinate dehydrogenase (sdhABCD), and 1-pyroline-5-carboxylate dehydrogenase (putA).

Escherichia bacteria having reduced α-ketoglutarate dehydrogenase (aKGDH) activity or deficient in the α-ketoglutarate dehydrogenase activity, and methods for obtaining them are described in U.S. Pat. Nos. 5,378,616 and 5,573,945. Further, methods for reducing or deleting the α-ketoglutarate dehydrogenase activity of Enterobacteriaceae bacteria such as Pantoea bacteria, Enterobacter bacteria, Klebsiella bacteria, and Erwinia bacteria are disclosed in U.S. Pat. Nos. 6,197,559, 6,682,912, 6,331,419, 8,129,151, and WO2008/075483. Specific examples of Escherichia bacteria having reduced α-ketoglutarate dehydrogenase activity or deficient in the α-ketoglutarate dehydrogenase activity include the following strains.

E. coli W3110sucA::Km^{r}

E. coli AJ12624 (FERM BP-3853)

E. coli AJ12628 (FERM BP-3854)

E. coli AJ12949 (FERM BP-4881)

E. coli W3110sucA::Km^{r} is a strain obtained by disrupting the α-ketoglutarate dehydrogenase gene (henceforth also referred to as “sucA gene”) of E. coli W3110. This strain is completely deficient in the α-ketoglutarate dehydrogenase activity.

Examples of L-glutamic acid-producing bacteria and parent strains that can be used to derive them also include Pantoea bacteria, such as the Pantoea ananatis AJ13355 strain (FERM BP-6614), Pantoea ananatis SC17 strain (FERM BP-11091), and Pantoea ananatis SC17(0) strain (VKPM B-9246). The AJ13355 strain is a strain isolated from soil in Iwata-shi, Shizuoka-ken, Japan as a strain that can proliferate in a low pH medium containing L-glutamic acid and a carbon source. The SC17 strain is a strain selected as a low phlegm-producing mutant strain from the AJ13355 strain (U.S. Pat. No. 6,596,517). The SC17 strain was deposited at National Institute of Advanced Industrial Science and Technology, International Patent Organism Depository (currently independent administrative agency, National Institute of Technology and Evaluation, International Patent Organism Depositary, #120, 2-5-8 Kazusakamatari, Kisarazu-shi, Chiba-ken, 292-0818, Japan) on Feb. 4, 2009, and assigned an accession number of FERM BP-11091. The AJ13355 strain was deposited at the independent administrative agency, National Institute of Advanced Industrial Science and Technology, International Patent Organism Depository (currently, independent administrative agency, National Institute of Technology and Evaluation, International Patent Organism Depositary, #120, 2-5-8 Kazusakamatari, Kisarazu-shi, Chiba-ken, 292-0818, Japan) on Feb. 19, 1998 and assigned an accession number of FERM P-16644. Then, the deposit was converted to an international deposit under the provisions of Budapest Treaty on Jan. 11, 1999, and assigned an accession number of FERM BP-6614.

Further, examples of L-glutamic acid-producing bacteria and parent strains that can be used to derive them also include Pantoea bacteria having reduced α-ketoglutarate dehydrogenase activity or deficient in the α-ketoglutarate dehydrogenase activity. Examples of such strains include AJ13356 (U.S. Pat. No. 6,331,419), which is an αKGDH-E1 subunit (sucA) gene-deficient strain of the AJ13355 strain, and the SC17sucA strain (U.S. Pat. No. 6,596,517), which is a sucA gene-deficient strain of the SC17 strain. The AJ13356 strain was deposited at the National Institute of Bioscience and Human-Technology, Agency of Industrial Science and Technology (currently, independent administrative agency, National Institute of Technology and Evaluation, International Patent Organism Depositary, #120, 2-5-8 Kazusakamatari, Kisarazu-shi, Chiba-ken, 292-0818, Japan) on Feb. 19, 1998, and assigned an accession number of FERM P-16645. Then, the deposit was converted into an international deposit under the provisions of the Budapest Treaty on Jan. 11, 1999, and assigned an accession number of FERM BP-6616. The SC17sucA strain was assigned a private number of AJ417, and deposited at the National Institute of Advanced Industrial Science and Technology, International Patent Organism Depositary (currently, independent administrative agency, National Institute of Technology and Evaluation, International Patent Organism Depositary, #120, 2-5-8 Kazusakamatari, Kisarazu-shi, Chiba-ken, 292-0818, Japan) on Feb. 26, 2004 under an accession number of FERM BP-08646.

The AJ13355 strain was identified as Enterobacter agglomerans when it was isolated, but it was recently reclassified as Pantoea ananatis on the basis of nucleotide sequencing of 16S rRNA and so forth. Therefore, although the AJ13355 and AJ13356 strains are deposited at the aforementioned depository as Enterobacter agglomerans, they are referred to as Pantoea ananatis in this specification.

Furthermore, examples of L-glutamic acid-producing bacteria and parent strains that can be used to derive them also include the Pantoea ananatis SC17sucA/RSFCPG+pSTVCB strain, AJ13601 strain, NP106 strain, and NA1 strain. The SC17sucA/RSFCPG+pSTVCB strain was obtained by introducing the plasmid RSFCPG containing the citrate synthase gene (gltA), phosphoenolpyruvate carboxylase gene (ppc), and glutamate dehydrogenase gene (gdhA) derived from Escherichia coli, and the plasmid pSTVCB containing the citrate synthase gene (gltA) derived from Brevibacterium lactofermentum, into the SC17sucA strain. The AJ13601 strain is a strain selected from the SC17sucA/RSFCPG+pSTVCB strain as a strain resistant to a high concentration of L-glutamic acid at a low pH. The NP106 strain is a strain obtained from the AJ13601 strain by curing the RSFCPG and pSTVCB plasmids. The AJ13601 strain was deposited at the independent administrative agency, National Institute of Advanced Industrial Science and Technology, International Patent Organism Depositary (currently, independent administrative agency, National Institute of Technology and Evaluation, International Patent Organism Depositary, #120, 2-5-8 Kazusakamatari, Kisarazu-shi, Chiba-ken, 292-0818, Japan) on Aug. 18, 1999, and assigned an accession number FERM P-17516. Then, the deposit was converted to an international deposit under the provisions of the Budapest Treaty on Jul. 6, 2000, and assigned an accession number FERM BP-7207.

Examples of L-glutamic acid-producing bacteria and parent strains that can be used to derive them also include strains in which both the α-ketoglutarate dehydrogenase (sucA) activity and the succinate dehydrogenase (sdh) activity are reduced or deleted (Japanese Patent Laid-open (KOKAI) No. 2010-041920). Specific examples of such strains include, for example, the sucAsdhA double-deficient strain of Pantoea ananatis NA1 strain (Japanese Patent Laid-open (KOKAI) No. 2010-041920).

Examples of L-glutamic acid-producing bacteria and parent strains that can be used to derive them also include auxotrophic mutant strains. Specific examples of auxotrophic mutant strains include, but are not particularly limited to, bacteria belonging to the genus Escherichia, such as E. coli VL334thrC⁺ (VKPM B-8961, EP 1172433). E. coli VL334 (VKPM B-1641) is an L-isoleucine and L-threonine auxotrophic strain having mutations in the thrC and ilvA genes (U.S. Pat. No. 4,278,765). E. coli VL334thrC⁺ is an L-isoleucine-auxotrophic L-glutamic acid-producing bacterium obtained by introducing a wild-type allele of the thrC gene into the VL334 strain. The wild-type allele of the thrC gene was transferred by the method of general transduction using a bacteriophage P1 grown on the wild-type E. coli K12 strain (VKPM B-7) cells.

Examples of L-glutamic acid-producing bacteria and parent strains that can be used to derive them also include strains having resistance to an aspartic acid analogue. Such strains can also be deficient in, for example, the α-ketoglutarate dehydrogenase activity. Examples of strains having resistance to an aspartic acid analogue and deficient in the α-ketoglutarate dehydrogenase activity include, for example, E. coli strains AJ13199 (FERM BP-5807, U.S. Pat. No. 5,908,768), FFRM P-12379, which additionally has a lowered L-glutamic acid-decomposing ability (U.S. Pat. No. 5,393,671), and AJ13138 (FERM BP-5565, U.S. Pat. No. 6,110,714).

Examples of L-glutamic acid-producing bacteria and parent strains that can be used to derive them also include strains modified so that the D-xylose-5-phosphate phosphoketolase activity and/or the fructose-6-phosphate phosphoketolase activity have/has been enhanced (Japanese Patent Laid-open (KOHYO) No. 2008-509661). Either one of the D-xylose-5-phosphate phosphoketolase activity and the fructose-6-phosphate phosphoketolase activity may be enhanced, or both may be enhanced. In this specification, D-xylose-5-phosphate phosphoketolase and fructose-6-phosphate phosphoketolase may be collectively referred to as phosphoketolase.

The D-xylose-5-phosphate phosphoketolase activity means an activity for converting xylose-5-phosphate into glycelaldehyde-3-phosphate and acetyl phosphate with consuming phosphoric acid to release one molecule of H₂O. This activity can be measured by the method described by Goldberg, M. et al. (Methods Enzymol., 9, 515-520 (1996)) or the method described by L. Meile (J. Bacteriol., 183:2929-2936 (2001)).

The fructose-6-phosphate phosphoketolase activity means an activity for converting fructose-6-phosphate into erythrose-4-phosphate and acetyl phosphate with consuming phosphoric acid to release one molecule of H₂O. This activity can be measured by the method described by Racker, E. (Methods Enzymol., 5, 276-280 (1962)) or the method described by L. Meile (J. Bacteriol., 183:2929-2936 (2001)).

<L-Glutamine-Producing Bacteria>

Examples of methods for imparting or enhancing L-glutamine-producing ability include, for example, a method of modifying a bacterium so that the bacterium has an increased activity or activities of one or more L-glutamine biosynthesis enzymes. Examples of such enzymes include, but are not particularly limited to, glutamate dehydrogenase (gdhA) and glutamine synthetase (glnA).

Examples of methods for imparting or enhancing L-glutamine-producing ability also include, for example, a method of modifying a bacterium so that the bacterium has a reduced activity or activities of one or more kinds of enzymes selected form the enzymes that catalyze a reaction branching away from the biosynthesis pathway of L-glutamine to generate a compound other than L-glutamine are reduced. Examples of such enzymes include, but not particularly limited to, glutaminase.

Examples of L-glutamic acid-producing bacteria and parent strains that can be used to derive them include a strain belonging to the genus Escherichia and having a mutant glutamine synthetase in which the tyrosine residue of position 397 is replaced with another amino acid residue (U.S. Patent Published Application No. 2003/0148474).

<L-Proline-Producing Bacteria>

Examples of L-proline-producing bacteria and parent strains that can be used to derive them include a strain in which the activity or activities of one or more L-proline biosynthetic enzymes have been enhanced. Examples of the enzyme involved in the L-proline biosynthesis include glutamate-5-kinase, γ-glutamylphosphate reductase, and pyroline-5-carboxylate reductase. For enhancing the activity of such an enzyme, for example, the proB gene encoding a glutamate kinase desensitized to feedback inhibition by L-proline (German Patent No. 3127361) can be used.

Examples of L-proline-producing bacteria and parent strains that can be used to derive them also include a strain with a reduced activity of an enzyme involved in decomposition of L-proline. Examples of such an enzyme include proline dehydrogenase and ornithine aminotransferase.

Specific examples of L-proline-producing bacteria and parent strains that can be used to derive them include, but are not particularly limited to, bacteria belonging to the genus Escherichia, such as E. coli NRRL B-12403 and NRRL B-12404 (British Patent No. 2075056), E. coli VKPM B-8012 (Russian Patent Application No. 2000124295), E. coli plasmid mutant strains described in German Patent No. 3127361, E. coli plasmid mutant strains described by Bloom F. R. et al. (The 15th Miami winter symposium, 1983, p. 34), and E. coli 702ilvA strain (VKPMB-8012), which is deficient in the ilvA gene, and can produce L-proline (EP 1172433).

<L-Tryptophan-Producing Bacteria, L-Phenylalanine-Producing Bacteria, and L-Tyrosine-Producing Bacteria>

Examples of methods for imparting or enhancing L-tryptophan-producing ability, L-phenylalanine-producing ability, and/or L-tyrosine-producing ability include, for example, a method of modifying a bacterium so that the bacterium has an increased activity or activities of one or more enzymes such as L-tryptophan, L-phenylalanine, and/or L-tyrosine biosynthesis enzymes.

Examples of enzymes common to the biosynthesis systems of these aromatic amino acids include, but are not particularly limited to, 3-deoxy-D-arabinoheptulosonate-7-phosphate synthase (aroG), 3-dehydroquinate synthase (aroB), shikimate dehydrogenase (aroE), shikimate kinase (aroL), 5-enolpyruvylshikimate-3-phosphate synthase (aroA), and chorismate synthase (aroC) (European Patent No. 763127). The expressions of the genes encoding these enzymes are controlled by the tyrosine repressor (tyrR), and the activities of these enzymes may be enhanced by deleting the tyrR gene (European Patent No. 763127).

Examples of the L-tryptophan biosynthesis enzymes include, but are not particularly limited to, anthranilate synthase (trpE), tryptophan synthase (trpAB), and phosphoglycerate dehydrogenase (serA). For example, by introducing a DNA containing the tryptophan operon, L-tryptophan-producing ability can be imparted or enhanced. Tryptophan synthase consists of α and β subunits encoded by the trpA and trpB genes, respectively. Since the anthranilate synthase is subject to feedback inhibition by L-tryptophan, a gene encoding this enzyme introduced with a mutation for desensitization to feedback inhibition may be used for enhancing the activity of that enzyme. Since the phosphoglycerate dehydrogenase is subject to feedback inhibition by L-serine, a gene encoding this enzyme that includes a mutation for desensitization to feedback inhibition may be used for enhancing the activity of that enzyme. Further, by increasing the expression of the operon (ace operon) that includes the maleate synthase gene (aceB), isocitrate lyase gene (aceA), and isocitrate dehydrogenase kinase/phosphatase gene (aceK), L-tryptophan-producing ability may be imparted or enhanced (WO2005/103275).

Examples of the L-phenylalanine biosynthesis enzymes include, but are not particularly limited to, chorismate mutase and prephenate dehydratase. The chorismate mutase and prephenate dehydratase are encoded by the pheA gene as a bifunctional enzyme. Since the chorismate mutase and prephenate dehydratase are subject to feedback inhibition by L-phenylalanine, genes encoding these enzymes that include a mutation for desensitization to feedback inhibition may be used for enhancing the activities of these enzymes.

Examples of the L-tyrosine biosynthesis enzymes include, but are not particularly limited to, chorismate mutase and prephenate dehydrogenase. The chorismate mutase and prephenate dehydrogenase are encoded by the tyrA gene as a bifunctional enzyme. Since the chorismate mutase and prephenate dehydrogenase are subject to feedback inhibition by L-tyrosine, genes encoding these enzymes that include a mutation for desensitization to feedback inhibition may be used for enhancing the activities of these enzymes.

The L-tryptophan, L-phenylalanine, and/or L-tyro sine-producing bacteria may be modified so that biosynthesis of an aromatic amino acid other than the objective aromatic amino acid is reduced. Further, the L-tryptophan, L-phenylalanine, and/or L-tyrosine-producing bacteria may be modified so that a by-product uptake system is enhanced. Examples of the by-product include aromatic amino acids other than the objective aromatic amino acid. Examples of genes encoding such a by-product uptake system include, for example, tnaB and mtr, which are genes encoding the L-tryptophan uptake system, pheP, which is a gene encoding the L-phenylalanine uptake system, and tyrP, which is a gene encoding the L-tyrosine uptake system (EP 1484410).

Specific examples of L-tryptophan-producing bacteria and parent strains that can be used to derive them include, but are not particularly limited to, bacteria belonging to the genus Escherichia, such as E. coli JP4735/pMU3028 (DSM10122) and JP6015/pMU91 (DSM10123), which have a mutant trpS gene encoding a partially inactivated tryptophanyl-tRNA synthetase (U.S. Pat. No. 5,756,345), E. coli SV164, which has a trpE allele encoding an anthranilate synthase desensitized to feedback inhibition by tryptophan, E. coli SV164 (pGH5), which has a serA allele encoding a phosphoglycerate dehydrogenase desensitized to feedback inhibition by serine and a trpE allele encoding an anthranilate synthase desensitized to feedback inhibition by tryptophan (U.S. Pat. No. 6,180,373), a strain introduced with a tryptophan operon containing a trpE allele encoding an anthranilate synthase desensitized to feedback inhibition by tryptophan (Japanese Patent Laid-open (Kokai) Nos. 57-71397 and 62-244382, U.S. Pat. No. 4,371,614), E. coli AGX17(pGX44) (NRRL B-12263) and AGX6(pGX50)aroP (NRRL B-12264), which are deficient tryptophanase (U.S. Pat. No. 4,371,614), E. coli AGX17/pGX50,pACKG4-pps, which has an increased phosphoenolpyruvate-producing ability (WO97/08333, U.S. Pat. No. 6,319,696). Examples of L-tryptophan-producing bacteria and parent strains that can be used to derive them also include strains belonging to the genus Escherichia having an increased activity of the protein encoded by the yedA or yddG gene (U.S. Patent Published Applications 2003/0148473 A1 and 2003/0157667 A1).

Specific examples of L-phenylalanine-producing bacteria and parent strains that can be used to derive them include, but are not particularly limited to, bacteria belonging to the genus Escherichia, such as E. coli AJ12739 (tyrA::Tn10, tyrR) (VKPM B-8197), which is defficient in the chorismate mutase-prephenate dehydrogenase and the tyrosine repressor (WO03/044191), E. coli HW1089 (ATCC 55371), which contains a mutant pheA34 gene encoding a chorismate mutase-prephenate dehydratase desensitized to feedback inhibition (U.S. Pat. No. 5,354,672), E. coli MWEC101-b (KR 8903681), E. coli NRRL B-12141, NRRL B-12145, NRRL B-12146, and NRRL B-12147 (U.S. Pat. No. 4,407,952). Specific examples of L-phenylalanine-producing bacteria and parent strains that can be used to derive them also include E. coli K-12 [W3110(tyrA)/pPHAB] (FERM BP-3566), E. coli K-12 [W3110(tyrA)/pPHAD] (FERM BP-12659), E. coli K-12 [W3110(tyrA)/pPHATerm] (FERM BP-12662), and E. coli K-12 AJ12604[W3110(tyrA)/pBR-aroG4, pACMAB] (FERM BP-3579), which contain a gene encoding a chorismate mutase-prephenate dehydratase desensitized to feedback inhibition (EP 488424 B1). Examples of L-phenylalanine-producing bacteria and parent strains that can be used to derive them further include strains belonging to the genus Escherichia having increased activity of the protein encoded by the yedA gene or the yddG gene (U.S. Patent Published Applications Nos. 2003/0148473A1 and 2003/0157667A1, WO03/044192).

Further, examples of methods for imparting or enhancing an L-amino acid-producing ability include, for example, a method of modifying a bacterium so that the bacterium has an increased activity for secreting an L-amino acid from a bacterial cell. Such an activity for secreting an L-amino acid can be increased by, for example, increasing expression of a gene encoding a protein responsible for secretion of an L-amino acid. Examples of genes encoding the proteins responsible for secretion of various amino acids include, for example, b2682 gene (ygaZ), b2683 gene (ygaH), b1242 gene (ychE), and b3434 gene (yhgN) (Japanese Patent Laid-open (KOKAI) No. 2002-300874).

Further, examples of methods for imparting or enhancing an L-amino acid-producing ability also include, for example, a method of modifying a bacterium so that the bacterium has an increased activity of a protein involved in the glycometabolism, or a protein involved in the energy metabolism.

Examples of the protein involved in the glycometabolism include proteins involved in uptake of saccharides and the glycolysis system enzymes. Examples of genes encoding a protein involved in the glycometabolism include glucose-6-phosphate isomerase gene (pgi, WO01/02542), phosphoenolpyruvate synthase gene (pps, EP 877090 A), phosphoenolpyruvate carboxylase gene (ppc, WO95/06114), pyruvate carboxylase gene (pyc, WO99/18228, EP 1092776 A), phosphoglucomutase gene (pgm, WO03/04598), fructose bisphosphate aldolase gene (pfkB, fbp, WO03/04664), pyruvate kinase gene (pykF, WO03/008609), transaldolase gene (talB, WO03/008611), fumarase gene (fum, WO01/02545), non-PTS sucrose uptake gene (csc, EP 149911 A), and sucrose assimilation gene (scrAB operon, WO90/04636).

Examples of genes encoding the proteins involved in the energy metabolism include the transhydrogenase gene (pntAB, U.S. Pat. No. 5,830,716) and cytochrome bo-type oxidase gene (cyoB, EP 1070376 A).

The genes that can be used for the breeding of the aforementioned L-amino acid-producing bacteria are not limited to genes having the aforementioned genetic information and genes having a known nucleotide sequence, and may be a variant thereof, so long as the original functions of the encoded proteins are not degraded. For example, the genes that can be used for the breeding of the L-amino acid-producing bacteria may be a gene encoding a protein having an amino acid sequence of a known protein, but including substitution, deletion, insertion, or addition of one or several amino acid residues at one or several positions. For the variants of genes and proteins, the descriptions for variants of the lcfA gene and LcfA protein mentioned later can be applied, mutatis mutandis.

<1-2> Introduction of lcfA Gene

A bacterium can be modified by introduction of the lcfA gene. The bacterium can be obtained by introducing the lcfA gene into a bacterium belonging to the family Enterobacteriaceae and having an L-amino acid-producing ability such as those described above. The bacterium can also be obtained by introducing the lcfA gene into a bacterium belonging to the family Enterobacteriaceae, and then imparting an L-amino acid-producing ability to the bacterium or enhancing an L-amino acid-producing ability of the bacterium. The bacterium may be a bacterium that has acquired an L-amino acid-producing ability by introduction of the lcfA gene. The modifications for constructing the bacterium can be performed in an arbitrary order.

Hereafter, the lcfA gene will be explained.

he “lcfA gene” refers to a gene coding for a protein having an activity of generating a fatty acyl-CoA from a long chain fatty acid, and taking it up through the inner membrane. The “activity of generating a fatty acyl-CoA from a long chain fatty acid, and taking it up through the inner membrane” is referred to as “LcfA activity”.

The LcfA activity can be measured as, for example, the activity for taking up a long chain fatty acid. The activity for taking up a long chain fatty acid can be measured by, for example, a known method (Schmelter, T. et al., 2004, J. Biol. Chem., 279:24163-24170). The LcfA activity can also be measured as an activity for generating a fatty acyl-CoA from a long chain fatty acid (fatty acyl-CoA synthetase activity). The fatty acyl-CoA synthetase activity can be measured by, for example, a known method (Black, P. N. et al., J. Biol. Chem., 267 (35):25513-20 (1992)).

Examples of the lcfA gene include the lcfA gene of Bacillus subtilis (J. Biol. Chem., Vol. 282, No. 8, pp. 5180-5194). The nucleotide sequence of the lcfA gene of Bacillus subtilis and the amino acid sequence of the protein encoded by this gene (LcfA protein) are shown as SEQ ID NOS: 17 and 18, respectively.

The lcfA gene may be a gene coding for a variant of the aforementioned LcfA protein, so long as it has the LcfA activity. Such a variant may be referred to as “conservative variant”. Examples of the conservative variant include, for example, homologues and artificially modified proteins of the aforementioned LcfA protein.

A homologue of the aforementioned lcfA gene is easily obtained from a public database by, for example, BLAST search or FASTA search using the aforementioned nucleotide sequences of the lcfA gene (SEQ ID NO: 17) as a query sequence. Further, a homologue of the aforementioned lcfA gene can be obtained by, for example, PCR using the chromosome of a bacterium or yeast as the template, and oligonucleotides prepared on the basis of such a known gene sequence thereof as primers.

The gene encoding a conservative mutant of the LcfA protein may be, for example, such a gene as mentioned below. That is, the lcfA gene may be a gene encoding a protein having the aforementioned amino acid sequence including substitution, deletion, insertion or addition of one or several amino acid residues at one or several positions, so long as it encodes a protein having the LcfA activity. In such a case, usually 70% or more, 80% or more, or 90% or more, of the LcfA activity can be maintained as compared to the protein that does not include addition, deletion, insertion or addition of one or several amino acid residues. Although the number of “one or several” may differ depending on the positions in the three-dimensional structure of the protein or types of amino acid residues, specifically, it can be 1 to 20, 1 to 10, 1 to 5, or 1 to 3.

The aforementioned substitution, deletion, insertion, or addition of one or several amino acid residues is a conservative mutation that maintains normal function of the protein. Typical examples of the conservative mutation are conservative substitutions. The conservative substitution is a mutation wherein substitution takes place mutually among Phe, Trp, and Tyr, if the substitution site is an aromatic amino acid; among Leu, Ile, and Val, if it is a hydrophobic amino acid; between Gln and Asn, if it is a polar amino acid; among Lys, Arg, and His, if it is a basic amino acid; between Asp and Glu, if it is an acidic amino acid; and between Ser and Thr, if it is an amino acid having a hydroxyl group. Examples of substitutions considered as conservative substitutions include, specifically, substitution of Ser or Thr for Ala, substitution of Gln, His, or Lys for Arg, substitution of Glu, Gln, Lys, His, or Asp for Asn, substitution of Asn, Glu, or Gln for Asp, substitution of Ser or Ala for Cys, substitution of Asn, Glu, Lys, His, Asp, or Arg for Gln, substitution of Gly, Asn, Gln, Lys, or Asp for Glu, substitution of Pro for Gly, substitution of Asn, Lys, Gln, Arg, or Tyr for His, substitution of Leu, Met, Val, or Phe for Ile, substitution of Ile, Met, Val, or Phe for Leu, substitution of Asn, Glu, Gln, His, or Arg for Lys, substitution of Ile, Leu, Val, or Phe for Met, substitution of Trp, Tyr, Met, Ile, or Leu for Phe, substitution of Thr or Ala for Ser, substitution of Ser or Ala for Thr, substitution of Phe or Tyr for Trp, substitution of His, Phe, or Trp for Tyr, and substitution of Met, Ile, or Leu for Val. Further, such substitution, deletion, insertion, addition, inversion, or the like of amino acid residues as mentioned above includes a naturally occurring mutation due to an individual difference, or a difference of species of a bacterium from which the gene is derived (mutant or variant).

Furthermore, the gene having such a conservative mutation as mentioned above may be a gene encoding a protein showing a homology of 80% or more, 90% or more, 95% or more, 97% or more, or 99% or more, to the total amino acid sequence mentioned above, and having the LcfA activity. In this specification, “homology” may mean “identity”.

Moreover, the lcfA gene may be a DNA that is able to hybridize under stringent conditions with a probe that can be prepared from a known gene sequence, such as a sequence complementary to the whole or a part of the aforementioned nucleotide sequence, and encodes a protein having the LcfA activity. The “stringent conditions” refer to conditions under which a so-called specific hybrid is formed, and a non-specific hybrid is not formed. Examples of the stringent conditions include those under which highly homologous DNAs hybridize to each other, for example, DNAs not less than 80% homologous, preferably not less than 90% homologous, more preferably not less than 95% homologous, still more preferably not less than 97% homologous, particularly preferably not less than 99% homologous, hybridize to each other, and DNAs less homologous than the above do not hybridize to each other, or conditions of washing of typical Southern hybridization, i.e., conditions of washing once, or 2 or 3 times, at a salt concentration and temperature corresponding to 1×SSC, 0.1% SDS at 60° C., 0.1×SSC, 0.1% SDS at 60° C., or 0.1×SSC, 0.1% SDS at 68° C.

The probe used for the aforementioned hybridization may be a part of a sequence that is complementary to the gene as described above. Such a probe can be prepared by PCR using oligonucleotides prepared on the basis of a known gene sequence as primers and a DNA fragment containing the nucleotide sequences as a template. For example, when a DNA fragment having a length of about 300 by is used as the probe, the washing conditions of the hybridization may be, for example, 50° C., 2×SSC and 0.1% SDS.

Further, the lcfA gene may be a gene in which an arbitrary codon is replaced with an equivalent codon, so long as the gene encodes a protein having the LcfA activity. For example, the lcfA gene may be modified so that it has optimal codons according to codon frequencies observed in a host to be used.

The above descriptions concerning variants of genes and proteins can also be applied mutatis mutandis to arbitrary proteins such as L-amino acid biosynthetic enzymes and transporters, and genes encoding them.

The lcfA gene can be introduced into a bacterium according to the method of increasing the copy number of a gene described in the section of “Methods for increasing activity of protein” mentioned later. A DNA fragment containing the lcfA gene can be obtained by, for example, PCR using a genomic DNA of a microorganism having the lcfA gene as the template. The obtained DNA fragment containing the lcfA gene may be introduced into, for example, the chromosome of a bacterium. The obtained DNA fragment containing the lcfA gene may also be introduced into a bacterium by, for example, ligating the DNA fragment with a vector that functions in the host bacterium to construct an expression vector of the lcfA gene, and transforming the host bacterium with the expression vector.

Bacteria belonging to the family Enterobacteriaceae may inherently have the fadD gene as a gene corresponding to the lcfA gene. The bacterium may be modified so that the expression of the fadD gene inherently harbored by the bacterium is attenuated, so long as the lcfA gene has been introduced. The bacterium may be further modified so that the expression of the fadD gene is increased, in addition to that the lcfA gene has been introduced.

<1-3> Other Modifications

The bacterium as described herein may have been further modified so that the fatty acid-assimilating ability is increased. Examples of such modification include attenuating the expression of the fadR gene, enhancing the expression of one or more genes, such as the fadL, fadE, fadD, fadB, and fadA genes, enhancing the expression of the cyoABCDE operon, and a combination of these (Japanese Patent Laid-open (KOKAI) No. 2011-167071).

The fadR gene encodes the negative transcription factor of the fad regulon (DiRusso, C. C. et al., 1992, J. Biol. Chem. 267:8685-8691; DiRusso, C. C. et al., 1993, Mol. Microbiol., 7:311-322). The fad regulon contains the fadL, fadE, fadD, fadB, and fadA genes, and these genes encode the proteins involved in fatty acid metabolism. The fadR gene and the fad regulon are found in, for example, bacteria belonging to the family Enterobacteriaceae. The fadR gene of the Escherichia coli K12 MG1655 strain corresponds to the sequence of positions 1234161 to 1234880 in the genome sequence of this strain (GenBank accession No. NC_{—}000913). The FadR protein of the Escherichia coli K12 MG1655 strain is registered as GenBank accession No. NP_{—}415705.

The fadL gene encodes a transporter of the outer membrane having an ability to take up a long chain fatty acid (Kumar, G. B. and Black, P. N., 1993, J. Biol. Chem., 268:15469-15476; Stenberg, F. et al., 2005, J. Biol. Chem., 280:34409-34419). The fadL gene of the Escherichia coli K12 MG1655 strain corresponds to the sequence of positions 2459328 to 2460668 in the genome sequence of this strain (GenBank accession No. NC_{—}000913). The FadL protein of the Escherichia coli K12 MG1655 strain is registered as GenBank accession No. NP_{—}416846.

The fadD gene encodes a protein that catalyzes the reaction for generating a fatty acyl-CoA from a long chain fatty acid (fatty acyl-CoA synthetase activity), and takes up it through the inner membrane (Dirusso, C. C. and Black, P. N., 2004, J. Biol. Chem., 279:49563-49566; Schmelter, T. et al., 2004, J. Biol. Chem., 279: 24163-24170). The fadD gene of the Escherichia coli K12 MG1655 strain corresponds to the complementary sequence of the sequence of positions 1886085 to 1887770 in the genome sequence of this strain (GenBank accession No. NC_{—}000913). The FadD protein of the Escherichia coli K12 MG1655 strain is registered as GenBank accession No. NP_{—}416319.

The fadE gene encodes a protein having the acyl-CoA dehydrogenase activity for catalyzing the reaction of oxidizing a fatty acyl-CoA (O'Brien, W. J. and Frerman, F. E., 1977, J. Bacteriol., 132:532-540; Campbell, J. W. and Cronan, J. E., 2002, J. Bacteriol., 184:3759-3764). The fadE gene of the Escherichia coli K12 MG1655 strain corresponds to the complementary sequence of the sequence of positions 240859 to 243303 in the genome sequence of this strain (GenBank accession No. NC_{—}000913). The FadE protein of the Escherichia coli K12 MG1655 strain is registered as GenBank accession No. NP_{—}414756.

The fadB gene encodes the a subunit of the fatty acid oxidation complex. The α subunit has four kinds of activities of enoyl-CoA hydratase, 3-hydroxyacyl-CoA dehydrogenase, 3-hydroxyacyl-CoA epimerase, and Δ3-cis-Δ2-trans-enoyl-CoA isomerase (Pramanik, A. et al., 1979, J. Bacteriol., 137:469-473; Yang, S. Y. and Schulz, H., 1983, J. Biol. Chem., 258:9780-9785). The fadB gene of the Escherichia coli K12 MG1655 strain corresponds to the complementary sequence of the sequence of positions 4026805 to 4028994 in the genome sequence of this strain (GenBank accession No. NC_{—}000913). The FadB protein of the Escherichia coli K12 MG1655 strain is registered as GenBank accession No. NP_{—}418288.

The fadA gene encodes the β subunit of the fatty acid oxidation complex. The β subunit has the 3-ketoacyl-CoA thiolase activity (Pramanik, A. et al., 1979, J. Bacteriol., 137: 469-473). The fadA gene of the Escherichia coli K12 MG1655 strain corresponds to the complementary sequence of the sequence of positions 4025632 to 4026795 in the genome sequence of this strain (GenBank accession No. NC_{—}000913). The FadA protein of the Escherichia coli K12 MG1655 strain is registered as GenBank accession No. YP_{—}026272.

The fadA and fadB genes form the fadBA operon (Yang, S. Y. et al., 1990, J. Biol. Chem., 265:10424-10429). Therefore, for example, the expression of the whole fadBA operon may be enhanced.

The cyoABCDE operon (cyo operon) encodes the cytochrome bo-type terminal oxidase complex as one of terminal oxidases. Precisely, the cyoB gene encodes the subunit I, cyoA gene encodes the subunit II, cyoC gene encodes the subunit III, cyoC gene encodes the subunit IV, and cyoE gene encodes a protein having the heme O synthase activity (Gennis, R. B. and Stewart, V., 1996, pp. 217-261, In F. D. Neidhardt (ed.), Escherichia coli and Salmonella Cellular and Molecular Biology/Second Edition, American Society for Microbiology Press, Washington, D.C; Chepuri et al., 1990, J. Biol. Chem., 265:11185-11192). The cyo operon is found in, for example, bacteria belonging to the family Enterobacteriaceae. The cyoABCDE genes of the Escherichia coli K12 MG1655 strain corresponds to the complementary sequences of the sequences of positions 449887 to 450834, 447874 to 449865, 447270 to 447884, 446941 to 447270, and 446039 to 446929 in the genome sequence of this strain (GenBank accession No. NC_{—}000913), respectively. The CyoABCDE proteins of the Escherichia coli K12 MG1655 strain are registered as GenBank accession Nos. NP_{—}414966, NP_{—}414965, NP_{—}414964, NP_{—}414963, and NP_{—}414962, respectively.

The bacterium as described herein may also be modified so that the activity of pyruvate synthase (also referred to as “PS”) and/or pyruvate:NADP⁺ oxidoreductase (also referred to as “PNO”) is increased (WO2009/031565).

The “pyruvate synthase” means an enzyme reversibly catalyzing the reaction of generating pyruvic acid from acetyl-CoA and CO₂ using the reduced ferredoxin or reduced flavodoxin as an electron donor (EC 1.2.7.1). PS is also referred to as pyruvate oxidoreductase, pyruvate ferredoxin oxidoreductase, or pyruvate flavodoxin oxidoreductase. The activity of PS can be measured according to, for example, the method of Yoon et al. (Yoon, K. S. et al., 1997, Arch. Microbiol., 167:275-279).

Examples of a gene encoding PS (PS gene) include PS genes of bacteria having the reductive TCA cycle such as Chlorobium tepidum and Hydrogenobacter thermophilus, PS genes of bacteria belonging to the family Enterobacteriaceae such as Escherichia coli, and PS genes of autotrophic methanogens such as Methanococcus maripaludis, Methanocaldococcus jannaschii, and Methanothermobacter thermautotrophicus.

The “pyruvate:NADP⁺ oxidoreductase” means an enzyme reversibly catalyzing the reaction of generating pyruvic acid from acetyl CoA and CO₂ using NADPH or NADH as an electron donor (EC 1.2.1.15). The pyruvate:NADP⁺ oxidoreductase is also referred to as pyruvate dehydrogenase. The activity of PNO can be measured by, for example, the method of Inui et al. (Inui, H., et al., 1987, J. Biol. Chem., 262:9130-9135).

Examples of a gene encoding PNO (PNO gene) include the PNO gene of Euglena gracilis, which is a photosynthetic eukaryotic microorganism and is also classified into protozoans (Nakazawa, M. et al., 2000, FEBS Lett., 479:155-156), the PNO gene of a protist, Cryptosporidium parvum (Rotte, C. et al., 2001, Mol. Biol. Evol., 18:710-720, GenBank Accession No. AB021127), and a PNO homologous gene of Bacillariophyta, Tharassiosira pseudonana (Ctrnacta, V. et al., 2006, J. Eukaryot. Microbiol., 53:225-231).

Enhancement of the PS activity can also be attained by, besides the methods of increasing the activities of proteins such as described later, improving supply of the electron donor required for the PS activity. For example, the PS activity can be enhanced by enhancing the activity of recycling ferredoxin or flavodoxin of the oxidized form to that of the reduced form, enhancing the ability to biosynthesize ferredoxin or flavodoxin, or combination of them (WO2009/031565).

Examples of a protein having the activity of recycling ferredoxin or flavodoxin of the oxidized form to that of the reduced form include ferredoxin NADP⁺ reductase. The “ferredoxin NADP⁺ reductase” means an enzyme that reversibly catalyzes the reaction of converting ferredoxin or flavodoxin of the oxidized form to that of the reduced form using NADPH as the electron donor (EC 1.18.1.2). Ferredoxin NADP⁺ reductase is also referred to as flavodoxin NADP⁺ reductase. The activity of ferredoxin NADP+ reductase can be measured by, for example, the method of Blaschkowski et al. (Blaschkowski, H. P. et al., 1982, Eur. J. Biochem., 123:563-569).

Examples of a gene encoding ferredoxin NADP⁺ reductase (ferredoxin NADP⁺ reductase gene) include the fpr gene of Escherichia coli, the ferredoxin NADP+ reductase gene of Corynebacterium glutamicum, and the NADPH-putidaredoxin reductase gene of Pseudomonas putida (Koga, H. et al., 1989, J. Biochem. (Tokyo) 106:831-836).

The ability to biosynthesize ferredoxin or flavodoxin can be enhanced by enhancing the expression of a gene encoding ferredoxin (ferredoxin gene) or a gene encoding flavodoxin (flavodoxin gene). The ferredoxin gene or flavodoxin gene is not particularly limited, so long as it encodes ferredoxin or flavodoxin that can be utilized by PS and the electron donor recycling system.

Examples of the ferredoxin gene include the fdx gene and yfhL gene of Escherichia coli, the fer gene of Corynebacterium glutamicum, and ferredoxin genes of bacteria having the reductive TCA cycle such as Chlorobium tepidum and Hydrogenobacter thermophilus. Examples of the flavodoxin gene include the fldA gene and fldB gene of Escherichia coli, and flavodoxin genes of bacteria having the reductive TCA cycle.

The aforementioned genes such as the fadR gene, fad regulon, cyoABCDE operon, PS gene, PNO gene, ferredoxin NADP+ reductase gene, ferredoxin gene, and flavodoxin gene are not limited to genes having the aforementioned genetic information and genes having a known nucleotide sequence, and may be a variant thereof, so long as the functions of the encoded proteins are not degraded. For example, the genes may be a gene encoding a protein having an amino acid sequence of a known protein, but including substitution, deletion, insertion, or addition of one or several amino acid residues at one or several positions. For variants of genes or proteins, the descriptions concerning variants of the lcfA gene gene and LcfA protein mentioned above can be applied, mutatis mutandis.

<1-4> Methods for Increasing Activity of Protein

Hereafter, methods for increasing the activity of a protein will be explained.

The expression “the activity of a protein is increased” means that the activity of the protein per cell is increased as compared with that of a non-modified strain such as a wild-type strain or parent strain. The phrase that “the activity of a protein is increased” can also mean “the activity of a protein is enhanced”. Specifically, the expression “the activity of a protein is increased” means that the number of molecules of the protein per cell is increased, and/or the function of each molecule of the protein is increased as compared with those of a non-modified strain. That is, the term “activity” in the expression “the activity of a protein is increased” is not limited to the catalytic activity of the protein, but may mean the transcription amount of a gene (the amount of mRNA) coding for the protein, or the translation amount of the gene (the amount of the protein). Although the degree of the increase in the activity of a protein is not particularly limited so long as the activity of the protein is increased as compared with a non-modified strain, the activity of the protein may be increased, for example, 1.5 times or more, 2 times or more, or 3 times or more, as compared with that of a non-modified strain. Further, the phrase that “the activity of a protein is increased” includes not only that the activity of an objective protein is increased in a strain inherently having the activity of the objective protein, but also that the activity of an objective protein is imparted to a strain not inherently having the activity of the objective protein. Further, so long as the activity of the protein is eventually increased, the activity of the objective protein inherently contained in a host may be attenuated and/or deleted, and then a preferred type of the protein may be introduced thereto.

The modification that increases the activity of a protein is attained by, for example, increasing the expression of a gene coding for the protein. The phrase that “the expression of a gene is increased” is also referred to as “the expression of a gene is enhanced”. The expression of a gene may be increased 1.5 times or more, 2 times or more, or 3 times or more, as compared with that observed in a non-modified strain. Further, the phrase that “the expression of a gene is increased” includes not only that the expression amount of a target gene is increased in a strain that inherently expresses the target gene, but also that the gene is introduced into a strain that does not inherently express the target gene, and expressed therein. That is, the phrase “the expression of a gene is increased” also means, for example, that the target gene is introduced into a strain that does not have the gene, and expressed therein.

The expression of a gene can be increased by, for example, increasing the copy number of the gene.

The copy number of a gene can be increased by introducing the gene into the chromosome of a host microorganism. A gene can be introduced into a chromosome by, for example, using homologous recombination (Miller, J. H., Experiments in Molecular Genetics, 1972, Cold Spring Harbor Laboratory). Only one copy, or two or more copies of a gene may be introduced. For example, by performing homologous recombination using a sequence which is present in multiple copies on a chromosome as a target, multiple copies of a gene can be introduced into the chromosome. Examples of such a sequence which is present in multiple copies on a chromosome include repetitive DNAs, and inverted repeats located at the both ends of a transposon. Alternatively, homologous recombination may be performed by using an appropriate sequence on a chromosome such as a gene unnecessary for production of the L-amino acid as a target. Homologous recombination can be performed by, for example, a method of using a linear DNA such as Red-driven integration (Datsenko, K. A., and Wanner, B. L., Proc. Natl. Acad. Sci. USA, 97:6640-6645 (2000)), a method of using a plasmid having a temperature sensitive replication origin, a method of using a plasmid capable of conjugative transfer, a method of using a suicide vector not having a replication origin that functions in a host, or a transduction method using a phage. Further, a gene can also be randomly introduced into a chromosome by using a transposon or Mini-Mu (Japanese Patent Laid-open (Kokai) No. 2-109985, U.S. Pat. No. 5,882,888, EP 805867 B1).

Introduction of a target gene into a chromosome can be confirmed by Southern hybridization using a probe having a sequence complementary to the whole gene or a part thereof, PCR using primers prepared on the basis of the sequence of the gene, or the like.

Further, the copy number of a target gene can also be increased by introducing a vector containing the gene into a host bacterium. For example, the copy number of a target gene can be increased by ligating a DNA fragment containing the target gene with a vector that functions in a host to construct an expression vector of the gene, and transforming the host with the expression vector. The DNA fragment containing the target gene can be obtained by, for example, PCR using the genomic DNA of a microorganism having the target gene as the template. As the vector, a vector autonomously replicable in the cell of the host bacterium can be used. The vector is preferably a multi-copy vector. Further, the vector preferably has a marker such as an antibiotic resistance gene for selection of transformant. The vector may be, for example, a vector derived from a bacterial plasmid, a vector derived from a yeast plasmid, a vector derived from a bacteriophage, cosmid, phagemid, or the like. Specific examples of vector autonomously replicable in cells of Escherichia coli include, for example, pUC19, pUC18, pHSG299, pHSG399, pHSG398, pACYC184, pBR322, pSTV29 (all of these are available from Takara Bio), pMW219 (NIPPON GENE), pTrc99A (Pharmacia), pPROK series vectors (Clontech), pKK233-2 (Clontech), pET series vectors (Novagen), pQE series vectors (QIAGEN), and the broad host spectrum vector RSF1010.

When a gene is introduced, it is sufficient that the gene is expressibly harbored by the bacterium as described herein. Specifically, it is sufficient that the gene is introduced so that it is expressed under control by a promoter sequence that functions in the bacterium. The promoter may be a promoter derived from the host, or a heterogenous promoter. The promoter may be the native promoter of the gene to be introduced, or a promoter of another gene. As the promoter, for example, such a stronger promoter as mentioned later may also be used.

The gene to be introduced is not particularly limited so long as it encodes a protein that functions in the host. The gene to be introduced may be a gene derived from the host, or may be a heterogenous gene.

Further, when two or more of genes are introduced, it is sufficient that each of the genes is expressibly harbored by the bacterium as described herein. For example, all the genes may be carried by a single expression vector or a chromosome. Further, the genes may be separately carried by two or more expression vectors, or separately carried by a single or two or more expression vectors and a chromosome. An operon constituted by two or more genes may also be introduced.

Further, the expression of a gene can be increased by improving the transcription efficiency of the gene. The transcription efficiency of a gene can be improved by, for example, replacing the promoter of the gene on a chromosome with a stronger promoter. The “stronger promoter” means a promoter providing an improved transcription of a gene compared with an inherently existing wild-type promoter of the gene. Examples of stronger promoters include, for example, the known high expression promoters such as T7 promoter, trp promoter, lac promoter, tac promoter, and PL promoter. Further, as the stronger promoter, a highly-active type of an existing promoter may also be obtained by using various reporter genes. For example, by making the −35 and −10 regions in a promoter region closer to the consensus sequence, the activity of the promoter can be enhanced (WO00/18935). Examples of highly active-type promoter include various tac-like promoters (Katashkina J I et al., Russian Federation Patent Application No. 2006134574) and pnlp8 promoter (WO2010/027045). Methods for evaluating the strength of promoters and examples of strong promoters are described in the paper of Goldstein et al. (Prokaryotic Promoters in Biotechnology, Biotechnol. Annu. Rev., 1, 105-128 (1995)), and so forth.

Further, the expression of a gene can also be increased by improving the translation efficiency of the gene. The translation efficiency of a gene can be improved by, for example, replacing the Shine-Dalgarno (SD) sequence (also referred to as ribosome binding site (RBS)) for the gene on a chromosome with a stronger SD sequence. The “stronger SD sequence” means a SD sequence that provides an improved translation of mRNA compared with the inherently existing wild-type SD sequence of the gene. Examples of stronger SD sequences include, for example, RBS of the gene 10 derived from phage T7 (Olins P. O. et al, Gene, 1988, 73, 227-235). Further, it is known that substitution, insertion, or deletion of several nucleotides in a spacer region between RBS and the start codon, especially in a sequence immediately upstream of the start codon (5′-UTR), significantly affects the stability and translation efficiency of mRNA, and hence, the translation efficiency of a gene can also be improved by modifying them.

In the present invention, sites that affect the gene expression, such as a promoter, SD sequence, and spacer region between RBS and the start codon, are also collectively called “expression control region”. An expression control region can be identified by using a promoter search vector or gene analysis software such as GENETYX. Such an expression control region can be modified by, for example, a method of using a temperature sensitive vector or the Red driven integration method (WO2005/010175).

The translation efficiency of a gene can also be improved by, for example, modifying codons. For example, in the case of heterogenous expression of a gene or the like, the translation efficiency of the gene can be improved by replacing a rare codon present in the gene with a synonymous codon more frequently used. Codons can be replaced by, for example, the site-specific mutation method for introducing an objective mutation into an objective site of DNA. Alternatively, a gene fragment in which objective codons are replaced may be totally synthesized. Frequencies of codons in various organisms are disclosed in the “Codon Usage Database” (http://www.kazusa.or.jp/codon; Nakamura, Y. et al, Nucl. Acids Res., 28, 292 (2000)).

Further, the expression of a gene can also be increased by amplifying a regulator that increases the expression of the gene, or deleting or attenuating a regulator that reduces the expression of the gene.

Such methods for increasing the gene expression as mentioned above may be used independently or in an arbitrary combination.

Further, the modification that increases the activity of an enzyme can also be attained by, for example, enhancing specific activity of the enzyme. An enzyme showing an enhanced specific activity can be obtained by, for example, searching various organisms. Further, a highly-active type of an existing enzyme may also be obtained by introducing a mutation into the existing enzyme. Enhancement of the specific activity may be independently used, or may be used in an arbitrary combination with such methods for enhancing gene expression as mentioned above.

The method for the transformation is not particularly limited, and conventionally known methods can be used. There can be used, for example, a method of treating recipient cells with calcium chloride so as to increase the permeability thereof for DNA, which has been reported for the Escherichia coli K-12 strain (Mandel, M. and Higa, A., J. Mol. Biol., 1970, 53, 159-162), and a method of preparing competent cells from cells which are in the growth phase, followed by transformation with DNA, which has been reported for Bacillus subtilis (Duncan, C. H., Wilson, G. A. and Young, F. E., Gene, 1977, 1:153-167). Alternatively, there can also be used a method of making DNA-recipient cells into protoplasts or spheroplasts, which can easily take up recombinant DNA, followed by introducing a recombinant DNA into the DNA-recipient cells, which is known to be applicable to Bacillus subtilis, actinomycetes, and yeasts (Chang, S. and Choen, S. N., 1979, Mol. Gen. Genet., 168:111-115; Bibb, M. J., Ward, J. M. and Hopwood, 0.A., 1978, Nature, 274:398-400; Hinnen, A., Hicks, J. B. and Fink, G. R., 1978, Proc. Natl. Acad. Sci. USA, 75:1929-1933).

An increase in the activity of a protein can be confirmed by measuring the activity of the protein.

An increase in the activity of a protein can also be confirmed by confirming an increase in the expression of a gene coding for the protein. An increase in the expression of a gene can be confirmed by measuring an increase in the transcription amount of the gene, or by measuring an increase in the amount of a protein expressed from the gene.

An increase in the transcription amount of a gene can be confirmed by comparing the amount of mRNA transcribed from the gene with that observed in a non-modified strain such as a wild-type strain or parent strain. Examples of the method for evaluating the amount of mRNA include Northern hybridization, RT-PCR, and so forth (Sambrook, J., et al., Molecular Cloning A Laboratory Manual/Third Edition, Cold spring Harbor Laboratory Press, Cold Spring Harbor (USA), 2001). The amount of mRNA may increase, for example, 1.5 times or more, 2 times or more, or 3 times or more, as compared with that of a non-modified strain.

An increase in the amount of a protein can be confirmed by Western blotting using antibodies (Molecular Cloning, Cold Spring Harbor Laboratory Press, Cold Spring Harbor (USA), 2001). The amount of the protein may increase, for example, 1.5 times or more, 2 times or more, or 3 times or more, as compared with that of a non-modified strain.

The aforementioned methods for increasing the activity of a protein can be applied to, in addition to the introduction of the lcfA gene, enhancement of the activity of an arbitrary protein such as L-amino acid biosynthesis enzymes and transporters, and enhancement of the expression of an arbitrary gene such as genes coding for the those arbitrary proteins, fad regulon, cyoABCDE operon, PS gene, and PNO gene.

<1-5> Method for Reducing Activity of Protein

The methods for reducing the activity of a protein will be explained below.

The expression “the activity of a protein is reduced” means that the activity of the protein per cell is decreased as compared with that of a non-modified strain such as a wild-type strain or parent strain, and includes that the activity has completely disappeared. Specifically, the expression “the activity of a protein is reduced” means that the number of molecules of the protein per cell is reduced, and/or the function of each molecule of the protein is reduced as compared with those of a non-modified strain. That is, the term “activity” in the expression “the activity of a protein is reduced” is not limited to the catalytic activity of the protein, but may mean the transcription amount of a gene (the amount of mRNA) coding for the protein or the translation amount of the protein (the amount of the protein). The phrase that “the number of molecules of the protein per cell is reduced” includes that the protein does not exist at all. The phrase that “the function of each molecule of the protein is reduced” includes that the function of each protein molecule completely disappears. Although the degree of the reduction in the activity of a protein is not particularly limited so long as the activity is reduced as compared with that observed in a non-modified strain, it may be reduced to, for example, 50% or less, 20% or less, 10% or less, 5% or less, or 0% of that observed in a non-modified strain.

The modification for reducing the activity of a protein can be attained by, for example, reducing the expression of a gene coding for the protein. The phrase that “the expression of a gene is reduced” includes that the gene is not expressed at all. The phrase that “the expression of a gene is reduced” is also referred to as “the expression of a gene is attenuated”. The expression of a gene may be reduced to 50% or less, 20% or less, 10% or less, 5% or less, or 0% of that observed in a non-modified strain.

The reduction in gene expression may be due to, for example, a reduction in the transcription efficiency, a reduction in the translation efficiency, or a combination of them. The expression of a gene can be reduced by modifying an expression control sequence of the gene such as a promoter and a Shine-Dalgarno (SD) sequence. When an expression control sequence is modified, one or more nucleotides, two or more nucleotides, or three or more nucleotides, of the expression control sequence are modified. Further, a part or the whole of an expression control sequence may be deleted. The expression of a gene can also be reduced by, for example, manipulating a factor responsible for expression control. Examples of the factor responsible for expression control include low molecules responsible for transcription or translation control (inducers, inhibitors, etc.), proteins responsible for transcription or translation control (transcription factors etc.), nucleic acids responsible for transcription or translation control (siRNA etc.), and so forth.

The modification for reducing the activity of a protein can also be attained by, for example, disrupting a gene coding for the protein. Disruption of a gene can be attained by, for example, deleting a part or the whole of the coding region of the gene on a chromosome. Furthermore, the whole of a gene including sequences upstream and downstream from the gene on a chromosome may be deleted. The region to be deleted may be any region such as an N-terminus region, an internal region, or a C-terminus region, so long as the activity of the protein can be reduced. Deletion of a longer region can usually more surely inactivate the gene. Further, it is preferred that reading frames of the sequences upstream and downstream from the region to be deleted are not the same.

Disruption of a gene can also be attained by, for example, introducing a mutation for an amino acid substitution (missense mutation), a stop codon (nonsense mutation), a frame shift mutation which adds or deletes one or two nucleotide residues, or the like into the coding region of the gene on a chromosome (Journal of Biological Chemistry, 272:8611-8617 (1997); Proceedings of the National Academy of Sciences, USA, 95 5511-5515 (1998); Journal of Biological Chemistry, 26 116, 20833-20839 (1991)).

Disruption of a gene can also be attained by, for example, inserting another sequence into a coding region of the gene on a chromosome. Site of the insertion may be in any region of the gene, and insertion of a longer region can usually more surely inactivate the gene. It is preferred that reading frames of the sequences upstream and downstream from the insertion site are not the same. The other sequence is not particularly limited so long as a sequence that reduces or eliminates the activity of the encoded protein is chosen, and examples thereof include, for example, a marker gene such as antibiotic resistance genes, and a gene useful for production of a heterologous protein.

Such modification of a gene on a chromosome as described above can be attained by, for example, preparing a deficient type gene in which a partial sequence of the gene is deleted so that it cannot produce a protein that can normally function, and transforming a bacterium with a recombinant DNA containing the deficient type gene to cause homologous recombination between the deficient type gene and the wild-type gene on a chromosome and thereby substitute the deficient type gene for the wild-type gene on the chromosome. In this procedure, if a marker gene selected according to the characteristics of the host such as auxotrophy is contained in the recombinant DNA, the operation becomes easy. The protein encoded by the deficient type gene has a conformation different from that of the wild-type protein, even if it is produced, and thus the function thereof is reduced or eliminated. Such gene disruption based on gene substitution utilizing homologous recombination has already been established, and there are methods of using a linear DNA such as a method called “Red driven integration” (Datsenko, K. A, and Wanner, B. L., Proc. Natl. Acad. Sci. USA, 97:6640-6645 (2000)), and a method utilizing the Red driven integration in combination with an excision system derived from λ phage (Cho, E. H., Gumport, R. I., Gardner, J. F., J. Bacteriol., 184:5200-5203 (2002)) (refer to WO2005/010175), a method of using a plasmid having a temperature sensitive replication origin, a method of using a plasmid capable of conjugative transfer, a method of utilizing a suicide vector not having a replication origin that functions in a host (U.S. Pat. No. 6,303,383, Japanese Patent Laid-open (Kokai) No. 05-007491), and so forth.

Modification for reducing the activity of a protein can also be attained by, for example, a mutagenesis treatment. Examples of the mutagenesis treatment include usual mutagenesis treatments including irradiation of X-ray or ultraviolet, and a treatment with a mutation agent such as N-methyl-N′-nitro-N-nitrosoguanidine (MNNG), ethyl methanesulfonate (EMS), and methyl methanesulfonate (MMS).

A reduction in the activity of a protein can be confirmed by measuring the activity of the protein.

A reduction in the activity of a protein can also be confirmed by measuring a reduction in the expression of a gene coding for the protein. A reduction in the expression of a gene can be confirmed by measuring a reduction in the transcription amount of the gene or a reduction in the amount of the protein expressed from the gene.

A reduction in the transcription amount of a gene can be confirmed by comparing the amount of mRNA transcribed from the gene with that observed in a non-modified strain. Examples of the method for evaluating the amount of mRNA include Northern hybridization, RT-PCR, and so forth (Molecular Cloning, Cold spring Harbor Laboratory Press, Cold Spring Harbor (USA), 2001). The amount of mRNA may be decreased to, for example, 50% or less, 20% or less, 10% or less, 5% or less, or 0%, of that observed in a non-modified strain.

A reduction in the amount of a protein can be confirmed by Western blotting using antibodies (Molecular Cloning, Cold Spring Harbor Laboratory Press, Cold Spring Harbor (USA) 2001). The amount of the protein may be decreased to, for example, 50% or less, 20% or less, 10% or less, 5% or less, or 0%, of that observed in a non-modified strain.

Disruption of a gene can be confirmed by determining nucleotide sequence of a part or the whole of the gene, restriction enzyme map, full length, or the like of the gene depending on the means used for the disruption.

The aforementioned methods for reducing the activity of a protein can be applied to reduction in the activity of an arbitrary protein such as enzymes that catalyze a reaction branching away from the biosynthetic pathway of an objective L-amino acid to generate a compound other than the objective L-amino acid and repressors of L-amino acid biosynthesis enzymes, and reduction in the expression of an arbitrary gene such as genes coding for those arbitrary proteins and the fadR gene.

<2>Method for Producing L-Amino Acid

The method as described herein is a method for producing an L-amino acid comprising culturing the bacterium as described herein in a medium containing a fatty acid, and collecting the L-amino acid from the medium. That is, according to the method as described herein, an L-amino acid can be produced by fermentation using a fatty acid as the carbon source.

The term “fatty acid” refers to a monovalent carboxylic acid of long chain hydrocarbon represented by the general formula CₙHₘ(n+1 and m+1 represent the number of carbon atoms and the number of hydrogen atoms contained in the fatty acid, respectively). There are a variety of fatty acids with varying number of carbons and varying degree of unsaturation. In general, a fatty acid having 12 or more carbon atoms is often referred to as a long chain fatty acid. It is also known that fatty acids are constituents of fat or oil, and the composition of fatty acid constituting fat or oil varies according to the types of fat or oil.

The fatty acid is not particularly limited so long as the bacterium of the present invention can utilize it as the carbon source. Examples of the fatty acid include, for example, lauric acid, myristic acid, palmitic acid, stearic acid, oleic acid, and linoleic acid. Among these, fatty acid(s) selected from lauric acid, myristic acid, palmitic acid, stearic acid, and oleic acid are preferred because these fatty acids are easily utilized by the bacterium of the present invention. Lauric acid (C₁₁H₂₃COOH) is a saturated fatty acid having 12 carbon atoms, and is contained in coconut oil and palm oil. Myristic acid (C₁₃H₂₇COOH) is a saturated fatty acid having 14 carbon atoms, and is contained in coconut oil and palm oil. Palmitic acid (C₁₅H₃₁COOH) is a saturated fatty acid having 16 carbon atoms, and is abundantly contained in vegetable fats and oils in general. Stearic acid (C₁₇H₃₅COOH) is a saturated fatty acid having 18 carbon atoms, and is abundantly contained in animal fats and vegetable oils. Oleic acid (C₁₇H₃₃COOH) is a monounsaturated fatty acid having 18 carbon atoms, and is abundantly contained in animal fats or vegetable oils. Linoleic acid (C₁₇H₃₁COOH) is a polyunsaturated fatty acid having 18 carbon atoms and two double bonds in the cis-configuration at positions 9 and 12, and is abundantly contained in vegetable oils such as safflower oil and corn oil. As the fatty acid, a single kind of fatty acid may be used, or two or more kinds of fatty acids may be used in combination. When two or more kinds of fatty acids are used, the mixing ratios of the fatty acids are not particularly limited, so long as the bacterium can utilize the fatty acids as the carbon source.

As the fatty acid, a pure fatty acid such as a purified fatty acid may be used, or a mixture containing a fatty acid and a component other than the fatty acid may be used. Examples of such a mixture include hydrolysates of fat or oil.

Fat or oil is an ester of fatty acid(s) and glycerol, and is also called triglyceride. Fat or oil is not particularly limited so long as it contains a fatty acid that can be utilized by the bacterium as the carbon source as a constituent, and it can be hydrolyzed. As fat or oil, those containing a fatty acid that can be utilized by the bacterium as the carbon source at a high content as a constituent are preferred. As fat or oil, there can be used fat or oil in any form including oils, which refer to those in a liquid state at ordinary temperature, and fats, which refer to those in a solid state at ordinary temperature. Furthermore, as fat or oil, there can be used fat or oil of any origin including animal fats and oils (including fish fats and oils) and vegetable fats and oils. Fat or oil can be used independently or as a combination of two or more kinds of them. As fat or oil, pure fat or oil such as purified fat or oil may be used, or a mixture containing fat or oil and a component other than fat or oil may be used. In the case of vegetable fat or oil, examples of such a mixture include, for example, a plant extract containing fat or oil and a fractionation product thereof containing fat or oil, such as oil cake. Oil cake is a by-product of the production process of vegetable oils, mainly produced in the acid removal treatment performed for removing free fatty acids in the purification process of vegetable oils, and it generally contains 40 to 70% of water, and 20 to 50% of fat or oil. In addition, crude glycerol produced in the production process of biodiesel fuel may contain several percent of fatty acid methyl esters, which constitute biodiesel fuel, and free fatty acids, and they can be fractionated and used.

Specific examples of animal fats and oils include, for example, butter, lard, beef tallow, mutton tallow, whale oil, sardine oil, and herring oil. Specific examples of vegetable fats and oils include, for example, palm oil, olive oil, rapeseed oil, soybean oil, rice bran oil, walnut oil, sesame oil, and peanut oil. Palm oil is oil that can be obtained from fruits of oil palm, and has come to be widely used as biodiesel fuel in recent years, and therefore the production amount thereof is increasing. Oil palm is a generic name for the plants classified into the genus Elaeis of the family Palmae. Crude palm oil generally refers to unrefined palm oil produced at oil mills, and such palm oil is traded as crude palm oil. In addition, microalgae that accumulate fat or oil are known (Chisti, Y., Biotechnol. Adv., 2007, 25: 294-306), and fat or oil extracted from alga cells can also be used. Alga cells also contain organic substances other than fat or oil, such as saccharides, proteins, and amino acids, and a mixture containing these substances can be hydrolyzed and used as the carbon source.

A hydrolysate of fat or oil can be obtained by hydrolyzing fat or oil. The hydrolysis can be performed, for example, chemically or enzymatically. As an industrial hydrolysis method, a continuous high temperature hydrolysis method in which fat or oil is brought into contact with water by countercurrent contacting at a high temperature (250 to 260° C.) under a high pressure (5 to 6 MPa) is commonly performed. A hydrolysis reaction performed at low temperature (about 30° C.) by using an enzyme is also industrially used (Jaeger, K. E. et al., 1994, FEMS Microbial. Rev., 15:29-63). As the aforementioned enzyme, lipases, which are enzymes that catalyze a hydrolysis reaction of fat or oil, can be used. Lipases are industrially important enzymes and used for various industrial applications (Hasan, F. et al., 2006, Enzyme and Microbiol. Technol., 39:235-251). A hydrolysate of fat or oil is obtained as a mixture of fatty acid(s) and glycerol. It is known that weight ratio of glycerol to the fatty acid(s) contained in a hydrolysate of common fat or oil such as palm oil is about 10%. The hydrolysate of fat or oil is not particularly limited so long as the hydrolysate contains a fatty acid. A hydrolysate of fat or oil can be used as it is, or a hydrolysate of fat or oil from or to which a desired component is removed or added can also be used. For example, a mixture of fatty acids obtained from a hydrolysate of fat or oil by removing glycerol may be used as the carbon source. Further, for example, a desired fatty acid may be obtained from a hydrolysate of fat or oil, and used as the carbon source.

The fatty acid may be in the form of free compound, a salt thereof, or a mixture of them. Examples of the salt include alkali metal salts such as sodium salt and potassium salt. Alkali metal salts of fatty acids are highly water-soluble, and maintained in water as micelles, and therefore they can be efficiently utilized by the bacterium.

It is also preferable to perform a treatment for promoting homogenization of the fatty acid to enhance the solubility of the fatty acid, so that the bacterium can more efficiently utilize the fatty acid.

Examples of the treatment for promoting homogenization include, for example, emulsification. Emulsification can be performed by, for example, adding an emulsification enhancer or a surfactant. Examples of the emulsification enhancer include, for example, phospholipids and sterols. As the surfactant, surfactants commonly used in the field of biology can be used. Examples of the surfactant include, as nonionic surfactants, for example, poly(oxyethylene) sorbitan fatty acid esters such as poly(oxyethylene) sorbitan monooleic acid ester (Tween 80); alkyl glucosides such as n-octyl β-D-glucoside; sucrose fatty acid esters such as sucrose stearic acid ester; polyglycerin fatty acid esters such as polyglycerin stearic acid ester; Triton X-100, polyoxyethylene(20) cetyl ether (Brij-58), and nonylphenol ethoxylate (Tergitol NP-40). Examples of the surfactant also include, as ampholytic surfactants, alkylbetaines such as N,N-dimethyl-N-dodecylglycine betaine.

Examples of the treatment for promoting homogenization include, for example, homogenizer treatment, homomixer treatment, ultrasonication, high pressure treatment, and high temperature treatment. Among these, homogenizer treatment and/or ultrasonication are preferred. Combinations of homogenizer treatment and/or ultrasonication and a treatment with a surfactant are more preferred.

The treatment for promoting homogenization is preferably carried out under an alkaline condition under which fatty acids are more stable. As the alkaline condition, pH not lower than 9 is preferred, and pH not lower than 10 is more preferred.

In the method as described herein, a fatty acid may be used as a sole carbon source, or may not be used as a sole carbon source. That is, in the method as described herein, another carbon source may be used together in addition to the fatty acid. Examples of the other carbon source include, but are not particularly limited to, saccharides such as glucose, fructose, sucrose, lactose, galactose, xylose, arabinose, blackstrap molasses, starch hydrolysates, and hydrolysates of biomass; organic acids such as fumaric acid, citric acid, and succinic acid; and alcohols such as ethanol, glycerol, and crude glycerol. When the other carbon source is used, the ratio of the fatty acid in the total carbon source may be, for example, 10% by weight or more, preferably 30% by weight or more, more preferably 50% by weight or more. The ratio of the fatty acid in the total carbon source may also be arbitrarily selected depending on the raw material to be used, and specifically, when the fatty acid and glucose are used as the carbon source, the ratio of the fatty acid based on the total amount of the fatty acid and glucose may be, for example, 2.5% by weight, 5% by weight, 10% by weight, 15% by weight, or 20% by weight. As the other carbon source, a single kind of carbon source may be used, or two or more kinds of carbon sources may be used in combination.

In the method as described herein, as the medium components, in addition to the carbon source, other components can be appropriately used. Examples of the components other than the carbon source include, for example, nitrogen source, sulfur source, phosphate source, and growth-promoting factors (components having growth-promoting activity).

Examples of the nitrogen source include ammonia, ammonium salts, nitrates, and urea. Examples of the ammonium salts include ammonium sulfate, ammonium carbonate, ammonium chloride, ammonium phosphate, and ammonium acetate. Ammonia gas and aqueous ammonia used for pH adjustment can also be used as the nitrogen source. Examples of the nitrogen source further include organic nitrogen sources such as peptone, yeast extract, meat extract, malt extract, corn steep liquor, and soybean hydrolysate. As the nitrogen source, a single kind of nitrogen source may be used, or two or more kinds of nitrogen sources may be used in combination.

Examples of the phosphate source include phosphoric acid salts such as potassium dihydrogenphosphate and dipotassium hydrogenphosphate, phosphoric acid polymers such as pyrophosphoric acid, and so forth. As the phosphate source, a single kind of phosphate source may be used, or two or more kinds of phosphate sources may be used in combination.

Examples of the sulfur source include inorganic sulfur compounds such as sulfates, thiosulfates, and sulfites, and sulfur-containing amino acids such as cysteine, cystine, and glutathione. Among these, ammonium sulfate is preferred. As the sulfur source, a single kind of sulfur source may be used, or two or more kinds of sulfur sources may be used in combination.

Examples of the growth-promoting factor include trace metals, amino acids, vitamins, and nucleic acids, as well as peptone, casamino acid, yeast extract, soybean protein degradation product, which contain the foregoing substances. Examples of the trace metals include iron, manganese, magnesium, and calcium. Examples of the vitamins include vitamin B₁, vitamin B₂, vitamin B₆, nicotinic acid, nicotinamide, and vitamin B₁₂. As the growth-promoting factor, a single kind of growth-promoting factor may be used, or two or more kinds of growth-promoting factors may be used in combination.

Further, when an auxotrophic mutant strain that requires an amino acid or the like for growth thereof is used, it is preferable to supplement the required nutrient to the medium. For example, in many of L-lysine-producing bacteria, the L-lysine biosynthetic pathway is enhanced, and L-lysine degrading ability is attenuated. Therefore, when such an L-lysine-producing bacterium is cultured, for example, one or more types of amino acids selected from L-threonine, L-homoserine, L-isoleucine, and L-methionine are preferably added to the medium.

The culture conditions are not particularly limited so long as the bacterium can proliferate, and an objective L-amino acid can be produced. The culture can be performed, for example, under usual conditions used for culturing bacteria such as Escherichia coli. The culture conditions can be appropriately set according to various conditions such as type of bacterium to be used, and type of amino acid to be produced.

The culture can be performed as batch culture, fed-batch culture, continuous culture, or a combination of these. The medium used at the time of the start of the culture is also referred to as “starting medium”. The medium supplied to a culture system (fermentation tank) in fed-batch culture or continuous culture is also referred to as “feed medium”. Further, to supply a medium to a culture system in fed-batch culture or continuous culture is also referred to as to “feed”.

The medium components, for example, the carbon source such as fatty acids, nitrogen source, sulfur source, phosphate source, and growth-promoting factor, may be contained in the starting medium, feed medium, or the both. The types of the components contained in the starting medium may be or may not be the same as the types of the components contained in the feed medium. The concentration of each component contained in the starting medium may be or may not be the same as the concentration of the component contained in the feed medium. Further, two or more kinds of feed media containing different types and/or different concentrations of components may be used. For example, when medium is intermittently fed a plurality of times, the types and/or concentrations of components contained in the feed media may be or may not be the same.

The concentration of the fatty acid in the medium is not particularly limited, so long as the bacterium can use the fatty acid as the carbon source. The concentration of the fatty acid in the medium may be, for example, 10 w/v % or lower, 5 w/v % or lower, 2 w/v % or lower. The concentration of the fatty acid in the medium may be, for example, 0.2 w/v % or higher, 0.5 w/v % or higher, or 1.0 w/v % or higher. The fatty acid may be contained in the starting medium, feed medium, or the both at a concentration within the range exemplified above.

When the fatty acid is contained in the feed medium, the fatty acid may be contained in the feed medium at such a concentration that the fatty acid concentration in the medium after feeding is, for example, 5 w/v % or lower, 2 w/v % or lower, or 1 w/v % or lower. When the fatty acid is contained in the feed medium, the fatty acid may be contained in the feed medium at such a concentration that the fatty acid concentration in the medium after feeding is, for example, 0.01 w/v % or higher, 0.02 w/v % or higher, or 0.05 w/v % or higher.

When the fatty acid is used as a sole carbon source, the fatty acid may be contained at a concentration within the range exemplified above. When another carbon source is used together, the fatty acid may also be contained at a concentration within the range exemplified above. When another carbon source is used together, the fatty acid may also be contained at a concentration within a range defined by appropriately modifying the range exemplified above on the basis of, for example, ratio of the fatty acid in the total carbon source, or the like.

The fatty acid may be or may not be contained at a constant concentration throughout the whole culture process. For example, the fatty acid may run short for a certain period of time. The term “run short” means that the amount of the fatty acid is smaller than the required amount, and it may means, for example, that the concentration in the medium is 0. The “certain period of time” may be, for example, a period corresponding to 10% or shorter, 20% or shorter, or 30% or shorter, of the entire culture period. It is preferred that another carbon source is present in a sufficient amount during the period in which the fatty acid runs short. Even if fatty acids run short in a certain period of time as described above, so long as there is a period of culture in a medium containing a fatty acid, culture performed under such a condition is included in the scope of the expression “culture of a bacterium in a medium containing a fatty acid”.

Concentration of the fatty acid can be measured by gas chromatography (Hashimoto, K. et al., Biosci. Biotechnol. Biochem., 1996, 70:22-30) or HPLC (Lin, J. T. et al., J. Chromatogr. A., 1998, 808:43-49).

The culture can be, for example, aerobically performed. For example, the culture can be performed as aeration culture or shaking culture. The oxygen concentration can be controlled to be, for example, 5 to 50%, or about 10%, of the saturated oxygen concentration. The temperature may be controlled to be, for example, 20 to 45° C., or 33 to 42° C. pH of the medium may be controlled to be, for example, 5 to 9. When pH decreases during the culture, for example, the culture may be performed in a medium to which calcium carbonate has been added beforehand, or the medium may be neutralized with an alkali such as ammonia gas and aqueous ammonia. By culturing the bacterium of the present invention under such conditions, for example, for about 10 to 120 hours, a marked amount of an L-amino acid is accumulated in the medium.

The culture may be performed as separate seed culture and main culture. In such a case, the culture conditions of the seed culture and the main culture may be the same or different. For example, both the seed culture and the main culture may be performed as batch culture. Alternatively, for example, the seed culture may be performed as batch culture, and the main culture may be performed as fed-batch culture or continuous culture.

In the fed-batch culture or continuous culture, feeding of the feed medium may be continued over the whole period of the culture or only for a partial period of the culture. In the fed-batch culture or continuous culture, feeding may be intermittently performed a plurality of times.

When feeding is intermittently performed a plurality of times, the feeding may be repeatedly started and stopped so that the period for one time of feeding is, for example, 30% or shorter, 20% or shorter, or 10% or shorter, of the total period of the feeding of the plurality of times.

Further, when feeding is intermittently performed a plurality of times, the carbon source concentration in the fermentation medium can also be automatically maintained at a low level by controlling the feeding so that the second and following feedings are started when the carbon source in the fermentation medium is depleted in the non-feeding periods immediately before the respective feeding periods (U.S. Pat. No. 5,912,113). Depletion of the carbon source can be detected on the basis of, for example, elevation of pH, or elevation of dissolved oxygen concentration.

In the continuous culture, extraction of the culture medium may be continued over the whole period of the culture or only for a partial period of the culture. Further, in the continuous culture, extraction of the culture medium may be intermittently performed a plurality of times. Extraction and feeding of the culture medium may be or may not be simultaneously performed. For example, after extracting the culture medium, feeding may be performed, or after performing feeding, the culture medium may be extracted. It is preferred that the volume of the culture medium to be extracted is equal to the volume of the medium to be fed. The expression “the volume of the culture medium to be extracted is equal to the volume of the medium to be fed equal volume” mentioned above may mean that the volume of the culture medium to be extracted is, for example, 93 to 107% of the volume of the medium to be fed.

When the culture medium is continuously extracted, the extraction is preferably started at the same time as or after the start of the feeding. For example, within 5 hours, 3 hours, or 1 hour, after the start of the feeding, the extraction can be started.

When the culture medium is intermittently extracted, it is preferred that, when the L-amino acid concentration reaches a predetermined level, a part of the culture medium is extracted to collect the L-amino acid, and then a fresh medium is fed to continue the culture.

Further, after the L-amino acid is collected from the extracted culture medium, the cells can be reused by recycling filtration residue containing the cells into the fermentation tank (French Patent No. 2669935).

Further, as a method for producing a basic amino acid such as L-lysine, there is known a method in which the basic amino acid is produced by fermentation using bicarbonate ions and/or carbonate ions as major counter ions of the basic amino acid (Japanese Patent Laid-open (KOKAI) No. 2002-65287, U.S. Patent Published Application No. 20020025564, EP 1813677 A).

In such a method, pH of the medium is controlled to be 6.5 to 9.0, or 6.5 to 8.0, during the culture, and 7.2 to 9.0 at the end of the culture, so that there is a culture period where 20 mM or more, 30 mM or more, or 40 mM or more, of bicarbonate ions and/or carbonate ions are present in the medium. In order to make bicarbonate and/or carbonate ions exist in the medium in an amount required as counter ions of the basic amino acid, it is preferable to control the internal pressure of the fermentation tank to be positive during the fermentation, to supply carbon dioxide gas into the culture medium, or to perform both the foregoing procedures.

The internal pressure of the fermentation tank during fermentation can be controlled to be positive by, for example, making the gas supply pressure higher than the exhaust pressure. By making the internal pressure of the fermentation tank positive, the carbon dioxide gas generated by fermentation dissolves in the culture medium to generate bicarbonate ions and/or carbonate ions, and these can serve as counter ions of the basic amino acid. The internal pressure of the fermentation tank can be, specifically, 0.03 to 0.2 MPa, 0.05 to 0.15 MPa, or 0.1 to 0.3 MPa, in terms of the gage pressure (pressure difference with respect to the atmospheric pressure). Furthermore, when carbon dioxide gas is supplied to the medium, for example, pure carbon dioxide gas or a mixed gas containing 5 volume % or more of carbon dioxide gas can be bubbled in the medium. The internal pressure in the fermentation tank, supply volume of carbon dioxide gas, and limited aeration volume can be determined by, for example, measuring pH of the medium, bicarbonate and/or carbonate ion concentration in the medium, or ammonia concentration in the medium.

In the conventional methods for producing a basic amino acid, a sufficient amount of ammonium sulfate and/or ammonium chloride is usually added to the medium in order to use sulfate ions and/or chloride ions as counter ions of the basic amino acid, or sulfuric acid decomposition products and/or hydrochloric acid decomposition products of proteins etc. are added to the medium as nutrient components. Therefore, large amounts of sulfate ions and/or chloride ions are present in the medium, and the concentration of the weakly acidic carbonate ions is extremely low, i.e., it is at a ppm order.

On the other hand, the aforementioned method (Japanese Patent Laid-open (KOKAI) No. 2002-65287, U.S. Patent Published Application No. 20020025564A, EP 1813677 A) is characterized in that the amounts of these sulfate ions and chloride ions to be used are reduced so that the carbon dioxide gas released by microorganism during fermentation is made to be dissolved in the medium, and used as counter ions.

That is, to reduce the amounts of sulfate ions and/or chloride ions to be used is one of the objects of the aforementioned method, and therefore, the total amount of sulfate ions or chloride ions contained in the medium is usually 700 mM or lower, 500 mM or lower, 300 mM or lower, 200 mM or lower, or 100 mM or lower. By lowering the concentrations of sulfate ions and/or chloride ions, it is made easier to make bicarbonate ions and/or carbonate ions exist in the medium. That is, in the aforementioned method, pH of the medium for making bicarbonate ions and/or carbonate ions exist in the medium in an amount required as the counter ions of the basic amino acid can be kept low as compared with that obtainable by the conventional methods.

Further, in the aforementioned method, it is preferred that the concentrations of anions other than bicarbonate ions and/or carbonate ions (also referred to as other anions) in the medium are low so long as they are present in amounts required for the growth of the basic amino acid-producing bacterium. Examples of the other anions include chloride ions, sulfate ions, phosphate ions, ionized organic acids, and hydroxide ions. The total molar concentration of these other anions is usually 900 mM or lower, 700 mM or lower, 500 mM or lower, 300 mM or lower, or 200 mM or lower.

In the aforementioned method, it is not necessary to add sulfate ions or chloride ions to the medium in an amount larger than that required for growth of the basic amino acid-producing bacterium. It is preferred that appropriate amounts of ammonium sulfate etc. are fed to the medium at an early stage of the culture, and the feeding is terminated in the middle of the culture. Alternatively, ammonium sulfate etc. may be fed to the medium with maintaining the balance with respect to the amounts of carbonate ions and/or bicarbonate ions dissolved in the medium. Further, ammonia may be fed to the medium as a nitrogen source of the basic amino acid. For example, when pH is controlled with ammonia, ammonia supplied in order to elevate pH may be used as a nitrogen source of the basic amino acid. Ammonia can be supplied independently or together with another gas.

Further, in the aforementioned method, it is preferable to control the total ammonia concentration in the medium to such a concentration that production of the basic amino acid is not inhibited. Examples of the total ammonia concentration at which “production of the basic amino acid is not inhibited” include an ammonia concentration providing yield and/or productivity corresponding to 50% or more, 70% or more, 90% or more, of the yield and/or productivity obtainable in the production of the basic amino acid under optimal conditions. Specifically, the total ammonia concentration in the medium can be 300 mM or lower, 250 mM or lower, or 200 mM or lower. The dissociation degree of ammonia decreases as the pH becomes higher. Non-dissociating ammonia is more toxic to bacteria compared with ammonium ions. Therefore, the upper limit of the total ammonia concentration depends on the pH of the culture medium. That is, as the pH of the culture medium increases, the acceptable total ammonia concentration decreases. Therefore, the total ammonia concentration at which “production of the basic amino acid is not inhibited” is preferably determined for each specific pH value. However, the total ammonia concentration range that is acceptable at the highest pH level during the culture can be used as the total ammonia concentration range throughout the entire culture period.

On the other hand, the total concentration of ammonia as a source of nitrogen required for growth of the basic amino acid-producing bacterium and production of the basic amino acid is not particularly limited, and can be appropriately determined, so long as depletion of ammonia does not continue during the culture, and decrease of productivity of the objective substance by the microorganism due to the shortage of the nitrogen source does not occur. For example, the ammonia concentration can be measured over time during the culture, and if ammonia in the medium is depleted, a small amount of ammonia can be added to the medium. Although the ammonia concentration after the addition of ammonia is not particularly limited, the total ammonia concentration can be, for example, 1 mM or higher, 10 mM or higher, or 20 mM or higher.

Further, in the aforementioned method, the medium may contain cations other than the basic amino acid. Examples of cations other than the basic amino acid include K, Na, Mg, and Ca originating in medium components. The total molar concentration of the cations other than the basic amino acid is preferably 50% or lower of the molar concentration of the total cations.

The L-amino acid can usually be collected from fermentation broth by a combination of conventionally known methods such as ion-exchange resin method (Nagai, H. et al., Separation Science and Technology, 39(16), 3691-3710), precipitation method, membrane separation method (Japanese Patent Laid-open Nos. 9-164323 and 9-173792), crystallization method (WO2008/078448, WO2008/078646), and other methods. When the L-amino acid accumulates in the cells, the cells can be disrupted with, for example, ultrasonic waves or the like, and the L-amino acid can be collected by the ion exchange resin method or the like from the supernatant obtained by removing the cells from the cell-disrupted suspension by centrifugation.

The collected L-amino acid can contain bacterial cells, medium components, moisture, and by-product metabolites of the bacterium in addition to the objective L-amino acid. Purity of the collected L-amino acid can be, for example, 50% or higher, 85% or higher, or 95% or higher (Japanese Patent No. 1214636, U.S. Pat. Nos. 5,431,933, 4,956,471, 4,777,051, 4,946,654, 5,840,358, 6,238,714, U.S. Patent Published Application No. 2005/0025878).

Furthermore, when the L-amino acid deposits in the medium, it can be collected by centrifugation, filtration, or the like. L-Amino acid deposited in the medium and L-amino acid dissolved in the medium can be isolated together after the L-amino acid dissolved in the medium is crystallized.

### EXAMPLES

The present invention will be more specifically explained with reference to the following examples. However, these examples should not be construed to limit the present invention in any meanings.

### Example 1

### Construction of L-lysine-Producing Escherichia coli Strain Introduced with fadLDEBA Genes

<1-1> Outline of Construction of Strain Introduced with fadLDEBA Genes

In this example, an L-lysine-producing Escherichia coli strain introduced with fadL, fadD, fadE, fadB, and fadA genes was constructed. These genes encode the enzymes of the β oxidation pathway of fatty acids (Clark, D. P. and Cronan Jr., J. E., 1996, pp. 343-357, In F.D. Neidhardt (ed.),), Escherichia coli and Salmonella Cellular and Molecular Biology/Second Edition, American Society for Microbiology Press, Washington, D.C.). The fadB and fadA genes form an operon consisting of fadBA.

As the parent strain of the gene-introduced strain, the L-lysine-producing Escherichia coli strain WC196ΔcadAΔldcC (AJ110692, henceforth also referred to as WC196LC), described in International Patent Publication WO2006/078039, was used. This strain is a strain obtained from the WC196 strain (FERM BP-5252) by disrupting the cadA gene and the ldcC gene. The WC196LC strain was deposited at the independent administrative agency, National Institute of Advanced Industrial Science and Technology, International Patent Organism Depositary (currently, the independent administrative agency, National Institute of Technology and Evaluation, International Patent Organism Depositary, #120, 2-5-8 Kazusakamatari, Kisarazu-shi, Chiba-ken, 292-0818, Japan) on Oct. 7, 2008 as an international deposit under the provisions of the Budapest Treaty, and assigned an accession number of FERM BP-11027.

The genes were introduced by constructing the fadEBA operon and the fadLD operon by PCR, and inserting them into the chromosome of WC196LC.

The fadEBA operon and the fadLD operon were first inserted into the chromosome of the Escherichia coli K-12 MG1655 strain by the method called “Red-driven integration”, which was first developed by Datsenko and Wanner (Datsenko, K. A, and Wanner, B. L., 2000, Proc. Natl. Acad. Sci. USA, 97, 12, 6640-6645). Then, by the P1 transduction using the obtained strain as the donor, the fadEBA operon and the fadLD operon were inserted into the chromosome of WC196LC. Further, the antibiotic resistance gene introduced into the constructed strain was removed by using the excision system derived from λ phage (Cho, E. H., Gumport, R. I., and Gardner, J. F., 2002, J. Bacteriol., 184:5200-5203). The specific construction procedure will be described below.

<1-2> Construction of Strain Introduced with fadEBA Operon

As an fadEBA operon sequence, there was constructed a DNA fragment containing the fadE gene, an att-cat-Ptac fragment, which was obtained by ligating a DNA fragment (au-cat) consisting of the attachment site of λ phage and chloramphenicol resistance gene ligated together and the tac promoter sequence (Ptac, Gene, 25(2-3), 167-178 (1983)), upstream from the fadE gene, and the fadBA gene downstream from the fadE gene. The att-cat-Ptac fragment can be constructed with reference to the construction of pMW118-attL-Cm-attR (WO2005/010175).

Specifically, PCR was performed by using the primers shown as SEQ ID NOS: 1 and 2, and the chromosomal DNA of the Escherichia coli K-12 MG1655 strain as the template to obtain an fadE fragment to be ligated with the att-cat-Ptac fragment and the fadBA gene. Further, PCR was performed by using the primers shown as SEQ ID NOS: 3 and 4, and the au-cat-Ptac fragment as the template to obtain the att-cat-Ptac fragment to be ligated to the 5′ end of the fadE fragment. Furthermore, PCR was performed by using the primers shown as SEQ ID NOS: 5 and 6, and the chromosomal DNA of the Escherichia coli K-12 MG1655 strain as the template to obtain an fadBA fragment to be ligated to the 3′ end of the fadE fragment. These three of the PCR products were purified, and ligated to the vector pMW 119 digested with BamHI by using In-Fusion Advantage PCR Cloning Kit (Clontech) to construct a plasmid pMW-att-cat-PtacfadEBA for amplification of the fadEBA operon sequence.

PCR was performed by using the primers shown as SEQ ID NOS: 7 and 8, and the plasmid pMW-att-cat-PtacfadEBA as the template to obtain an att-cat-PtacfadEBA fragment for introducing the fadEBA operon into the genome of the Escherichia coli K-12 MG1655 strain at the site of the yciQ gene, of which the function is unknown.

The obtained att-cat-PtacfadEBA fragment was inserted into the chromosome of the Escherichia coli K-12 MG1655 strain at the site of the yciQ gene by the Red-driven integration method. A candidate strain in which the intended gene substitution occurred was selected on the basis of chloramphenicol resistance. Occurrence of the intended gene substitution in the candidate strain was confirmed by PCR. The obtained fadEBA operon-introduced strain was designated as MG1655ΔyciQ::att-cat-PtacfadEBA.

P1 transduction was performed with the WC196LC strain by using the obtained MG1655ΔyciQ::att-cat-PtacfadEBA as the donor to construct a strain corresponding to WC196LC in which the fadEBA operon was inserted into the chromosome at the site of the yciQ gene. A candidate strain in which the intended gene substitution occurred was selected on the basis of chloramphenicol resistance. Occurrence of the intended gene substitution in the candidate strain was confirmed by PCR. The obtained fadEBA operon-introduced strain was designated as WC196LCΔyciQ::att-cat-PtacfadEBA.

Then, in order to remove the au-cat gene, the helper plasmid pMW-intxis-ts (Japanese Patent Laid-open (KOKAI) No. 2005-058227) was used. pMW-intxis-ts is a plasmid carrying a gene encoding the integrase (Int) of λ phage and a gene encoding excisionase (Xis), and having temperature-sensitive replication ability.

Competent cells of the WC196LCΔyciQ::att-cat-PtacfadEBA strain obtained above were prepared in a conventional manner, transformed with the helper plasmid pMW-intxis-ts, and plate-cultured at 30° C. on the LB agar medium containing 100 mg/L ampicillin, and ampicillin-resistant strains were selected. Then, the strains were subcultured at 42° C. on the LB agar medium in order to remove the pMW-intxis-ts plasmid, and the obtained colonies were examined for the ampicillin resistance and chloramphenicol resistance to obtain a strain in which att-cat and pMW-intxis-ts had been removed. This strain was designated as WC196LCPtacfadEBA strain.

<1-3> Construction of Strain Introduced with fadLD Operon and fadEBA Operon

As an fadLD operon sequence, there was constructed a DNA fragment containing the fadL gene, the tac promoter sequence and the ribosome binding site (RBS) derived from the T7 phage 10 gene upstream sequence (Gene, 73, 227-235 (1988)) upstream of the fadL gene, and the ribosome binding site (RBS) derived from the T7 phage 10 gene upstream sequence and the fadD gene downstream of the fadL gene.

Specifically, PCR was performed by using the primers shown as SEQ ID NOS: 9 and 10, and the chromosomal DNA of the Escherichia coli K-12 MG1655 strain as the template to obtain an fadL fragment to be ligated with the att-cat-Ptac fragment and the fadD gene. Further, PCR was performed by using the primers shown as SEQ ID NOS: 11 and 12, and the att-cat-Ptac fragment as the template to obtain the att-cat-Ptac fragment to be ligated to the 5′ end of the fadL fragment. Furthermore, PCR was performed by using the primers shown as SEQ ID NOS: 13 and 14, and the chromosomal DNA of the Escherichia coli K-12 MG1655 strain as the template to obtain an fadD fragment to be ligated to the 3′ end of the fadL fragment. When the fadD fragment was obtained, the original start codon sequence ttg of fadD in the chromosomal DNA sequence of the Escherichia coli K-12 MG1655 strain was replaced with atg. These three of the PCR products were purified, and ligated to the vector pMW119 digested with BamHI by using In-Fusion Advantage PCR Cloning Kit (Clontech) to construct a plasmid pMW-att-cat-PtacfadLD for amplification of the fadLD operon sequence.

PCR was performed by using the primers shown as SEQ ID NOS: 15 and 16, and the plasmid pMW-att-cat-PtacfadLD as the template to obtain an att-cat-PtacfadLD fragment for introducing the fadLD operon into the genome of the Escherichia coli K-12 MG1655 strain at the site of the yegD gene, of which the function is unknown.

The obtained att-cat-PtacfadLD fragment was inserted into the chromosome of the Escherichia coli K-12 MG1655 strain at the site of the yegD gene by the Red-driven integration method. A candidate strain in which the intended gene substitution occurred was selected on the basis of chloramphenicol resistance. Occurrence of the intended gene substitution in the candidate strain was confirmed by PCR. The obtained fadLD operon-introduced strain was designated as MG1655ΔyegD::att-cat-PtacfadLD.

P1 transduction was performed with the WC196LCPtacfadEBA strain by using the obtained MG1655ΔyegD::att-cat-PtacfadLD as the donor to construct a strain corresponding to the WC196LCPtacfadEBA strain in which the fadLD operon was inserted into the chromosome at the site of the yegD gene. A candidate strain in which the intended gene substitution occurred was selected on the basis of chloramphenicol resistance. Occurrence of the intended gene substitution in the candidate strain was confirmed by PCR. The obtained fadLD operon-introduced strain was designated as WC196LCPtacfadEBAΔyegD::att-cat-PtacfadLD.

Then, in order to remove the au-cat gene, competent cells of the WC196LCPtacfadEBAΔyegD::att-cat-PtacfadLD strain obtained above were prepared in a conventional manner, transformed with the helper plasmid pMW-intxis-ts, and plate-cultured at 30° C. on the LB agar medium containing 100 mg/L ampicillin, and ampicillin-resistant strains were selected. Then, the strains were subcultured at 42° C. on the LB agar medium in order to remove the pMW-intxis-ts plasmid, and the obtained colonies were examined for the ampicillin resistance and chloramphenicol resistance to obtain a strain in which au-cat and pMW-intxis-ts had been removed. This strain was designated as WC196LCPtacfadEBAPtacfadLD strain.

<1-4> Introduction of Plasmid for Lysine Production into WC196LCPtacfadEBAPtacfadLD Strain

The WC196LCPtacfadEBAPtacfadLD strain was transformed with the plasmid pCABD2 (WO95/16042) for lysine production carrying the dapA, dapB, lysC, and ddh genes in a conventional manner to obtain a WC196LCPtacfadEBAPtacfadLD/pCABD2 strain.

The obtained WC196LCPtacfadEBAPtacfadLD/pCABD2 strain was cultured at 37° C. in the LB medium containing 20 mg/L of streptomycin until OD600 became about 0.3. Then, a 40% glycerol solution was added to the culture broth in the same volume as the culture broth, and the mixture was stirred, then divided into appropriate volumes, and preserved at −80° C. to obtain glycerol stocks.

### Example 2

### Construction of Escherichia coli L-lysine-Producing Strain Introduced with lcfA Gene and fadLEBA Genes

<2-1> Outline of Construction of lcfA Gene and fadLEBA Gene-Introduced Strain

As the fadD gene of Bacillus subtilis, the lcfA gene has been reported (J. Biol. Chem., Vol. 282, No. 8, pp. 5180-5194). The entire nucleotide sequence of the Bacillus subtilis chromosome has already been clarified (Nature, 390:249-56 (1997)), and the nucleotide sequence of the lcfA gene is reported in this reference. The nucleotide sequence of the lcfA gene is shown as SEQ ID NO: 17, and the amino acid sequence encoded by the lcfA gene is shown as SEQ ID NO: 18. First, the fadLlcfA operon was constructed on the basis of the nucleotide sequence of the lcfA gene, and inserted into the chromosome of the MG1655 strain by the Red-driven integration method. Then, the fadLlcfA operon was inserted into the chromosome of WC196LCPtacfadEBA by P1 transduction using the MG1655 strain in which the fadLlcfA operon was inserted into the chromosome as the donor. Further, the antibiotic resistance gene incorporated into the chromosome of the constructed strain was removed with an excision system derived from λ phage. Specific procedure of the construction is described below.

<2-2> Construction of Strain Introduced with lcfA Gene and fadLEBA Genes

As an fadLlcfA operon sequence, there was constructed a DNA fragment containing the fadL gene, the tac promoter sequence and the ribosome binding site (RBS) derived from the T7 phage 10 gene upstream sequence (Gene, 73, 227-235 (1988)) upstream of the fadL gene, and the ribosome binding site (RBS) derived from the T7 phage 10 gene upstream sequence and the lcfA gene downstream of the fadL gene.

Specifically, PCR was performed by using the primers shown as SEQ ID NOS: 19 and 20, and the chromosomal DNA of the Escherichia coli K-12 MG1655 strain as the template to obtain an fadL fragment to be ligated with the att-cat-Ptac fragment and the lcfA gene. Further, PCR was performed by using the primers shown as SEQ ID NOS: 21 and 22, and the att-cat-Ptac fragment as the template to obtain the att-cat-Ptac fragment to be ligated to the 5′ end of the fadL fragment. Furthermore, PCR was performed by using the primers shown as SEQ ID NOS: 23 and 24, and the chromosomal DNA of the Bacillus subtilis 168M strain as the template to obtain an lcfA fragment to be ligated to the 3′ end of the fadL fragment. These three of the PCR products were purified, and ligated to the vector pMW 119 digested with BamHI by using In-Fusion Advantage PCR Cloning Kit (Clontech) to construct a plasmid pMW-att-cat-PtacfadLlcfA for amplification of the fadLlcfA operon sequence.

PCR was performed by using the primers shown as SEQ ID NOS: 25 and 26, and the plasmid pMW-att-cat-PtacfadLlcfA as the template to obtain an att-cat-PtacfadLlcfA fragment for introducing the fadLlcfA operon into the genome of the Escherichia coli K-12 MG1655 strain at the site of the yegD gene, of which the function is unknown.

The obtained att-cat-PtacfadLlcfA fragment was inserted into the chromosome of the Escherichia coli K-12 MG1655 strain at the site of the yegD gene by the Red-driven integration method. A candidate strain in which the intended gene substitution occurred was selected on the basis of chloramphenicol resistance. Occurrence of the intended gene substitution in the candidate strain was confirmed by PCR. The obtained fadLlcfA operon-introduced strain was designated as MG1655ΔyegD::att-cat-PtacfadLlcfA.

P1 transduction was performed with the WC196LCPtacfadEBA strain by using the obtained MG1655ΔyegD::att-cat-PtacfadLlcfA as the donor to construct a strain corresponding to the WC196LCPtacfadEBA strain in which the fadLlcfA operon was inserted into the chromosome at the site of the yegD gene. A candidate strain in which the intended gene substitution occurred was selected on the basis of chloramphenicol resistance. Occurrence of the intended gene substitution in the candidate strain was confirmed by PCR. The obtained fadLlcfA operon-introduced strain was designated as WC196LCPtacfadEBAΔyegD::att-cat-PtacfadLlcfA.

Then, in order to remove the au-cat gene, competent cells of the WC196LCPtacfadEBAΔ.yegD::att-cat-PtacfadLlcfA strain obtained above were prepared in a conventional manner, transformed with the helper plasmid pMW-intxis-ts, and plate-cultured at 30° C. on the LB agar medium containing 100 mg/L ampicillin, and ampicillin-resistant strains were selected. Then, the strains were subcultured at 42° C. on the LB agar medium in order to remove the pMW-intxis-ts plasmid, and the obtained colonies were examined for the ampicillin resistance and chloramphenicol resistance to obtain a strain in which au-cat and pMW-intxis-ts had been removed. This strain was designated as WC196LCPtacfadEBAPtacfadLlcfA strain.

<2-3> Introduction of Plasmid for Lysine Production into WC196LCPtacfadEBAPtacfadLlcfA Strain

The WC196LCPtacfadEBAPtacfadLlcfA strain was transformed with the plasmid pCABD2 (WO95/16042) for lysine production carrying the dapA, dapB, lysC, and ddh genes in a conventional manner to obtain a WC196LCPtacfadEBAPtacfadLlcfA/pCABD2 strain.

The obtained WC196LCPtacfadEBAPtacfadLlcfA/pCABD2 strain was cultured at 37° C. in the LB medium containing 20 mg/L of streptomycin until OD600 became about 0.3. Then, a 40% glycerol solution was added to the culture broth in the same volume as the culture broth, and the mixture was stirred, then divided into appropriate volumes, and preserved at −80° C. to obtain glycerol stocks.

### Example 3

### L-Lysine Production Using Strain Introduced with lcfA Gene and fadLEBA Genes

The glycerol stocks of the WC196LCPtacfadEBAPtacfadLlcfA/pCABD2 strain, WC196LCPtacfadEBAPtacfadLfadD/pCABD2 strain, and the control strain WC196LC/pCABD2 (WO2006/078039) were thawed, 100 μL of each was uniformly applied to an LB agar medium plate containing 20 mg/L of streptomycin, and culture was performed at 37° C. for 24 hours. Then, the cells corresponding to about ⅛, of the plate were inoculated into the fermentation medium (40 mL) mentioned below, containing 60 mg/L of streptomycin and contained in a 500 mL-volume conical flask, and cultured at 37° C. for 42 hours on a reciprocal shaking culture machine. The main culture was performed in duplicate for each strain. As the carbon source for the main culture, 30 g/L of glucose and 4 g/L of sodium oleate, or 20 g/L of glucose and 3 g/L of sodium oleate were used. Further, as an emulsification enhancer, poly(oxyethylene) sorbitan monooleic acid ester (Tween 80, Nakarai-Tesque) was added at a final concentration of 0.5% (w/v). It was separately confirmed that the strains could not assimilate Tween 80. The medium composition used for the culture is shown below.

Escherichia bacterium L-lysine Production Medium

### <Carbon Source>

| | |
|---|---|
| Glucose | 30 g/L |
| Sodium oleate | 4 g/L |
| or | |
| Glucose | 20 g/L |
| Sodium oleate | 3 g/L |

### <Other Components>

| | |
|---|---|
| (NH₄)₂SO₄ | 24 g/L |
| KH₂PO₄ | 1 g/L |
| MgSO₄•7H₂O | 1 g/L |
| FeSO₄•7H₂O | 0.01 g/L |
| MnSO₄•7H₂O | 0.008 g/L |
| Yeast Extract | 2 g/L |
| Tween 80 | 5 g/L |
| CaCO₃ (Japanese pharmacopoeia) | 22.5 g/L |

The medium was adjusted to pH 7.0 with KOH, and autoclaved at 120° C. for 20 minutes, provided that the carbon sources and MgSO₄.7H₂O were separately sterilized, and then mixed with the other components. CaCO₃ was added after hot air sterilization.

After the culture for 42 hours, the amount of L-lysine contained in the culture supernatant was measured with a biosensor BF-5 (Oji Scientific Instruments). The degree of the growth was measured on the basis of the turbidity (OD600) for the medium diluted with a 0.5% Tween solution.

The averages of the results obtained by using 30 g/L of glucose and 4 g/L of sodium oleate are shown in Table 1, and the averages of the results obtained by using 20 g/L of glucose and 3 g/L of sodium oleate are shown in Table 2. In both cases, the L-lysine-producing strain introduced with lcfA gene and fadLEBA genes (WC196LCPtacfadEBAPtacfadLlcfA/pCABD2) showed significantly higher L-lysine production than the control strain (WC196LC/pCABD2), and the L-lysine-producing strain introduced with the fadLDEBA genes (WC196LCPtacfadEBAPtacfadLfadD/pCABD2).

TABLE 1
Results of culture of L-lysine-producing strain introduced with lcfA gene
and fadLEBA genes performed by using 30 g/L of glucose and 4 g/L of
sodium oleate as carbon sources

| Strain | O.D. | L-Lysine (g/L) |
|---|---|---|
| WC196LC/pCABD2 | 18.7 | 14.1 |
| WC196LCPtacfadEBAPtacfadLD/pCABD2 | 20.1 | 14.2 |
| WC196LCPtacfadEBAPtacfadLlcfA/pCABD2 | 18.1 | 14.5 |

TABLE 2
Results of culture of L-lysine-producing strain introduced with lcfA gene
and fadLEBA genes performed by using 20 g/L of glucose and 3 g/L of
sodium oleate as carbon sources

| Strain | O.D. | L-Lysine (g/L) |
|---|---|---|
| WC196LC/pCABD2 | 14.2 | 10.6 |
| WC196LCPtacfadEBAPtacfadLD/pCABD2 | 14.1 | 10.3 |
| WC196LCPtacfadEBAPtacfadLlcfA/pCABD2 | 12.7 | 10.9 |

### INDUSTRIAL APPLICABILITY

According to the present invention, an L-amino acid-producing ability of a bacterium can be improved in the case of using a fatty acid as the carbon source, and an L-amino acid can be efficiently produced by using a fatty acid as the carbon source.

### EXPLANATION OF SEQUENCE LISTING

SEQ ID NOS: 1 and 2, PCR primers for amplification of fadE gene fragment

SEQ ID NOS: 3 and 4, PCR primers for amplification of att-cat-Ptac fragment

SEQ ID NOS: 5 and 6, PCR primers for amplification of fadBA gene fragment

SEQ ID NOS: 7 and 8, PCR primers for amplification of att-cat-PtacfadEBA gene fragment

SEQ ID NOS: 9 and 10, PCR primers for amplification of fadL gene fragment

SEQ ID NOS: 11 and 12, PCR primers for amplification of att-cat-Ptac fragment

SEQ ID NOS: 13 and 14, PCR primers for amplification of fadD gene fragment

SEQ ID NOS: 15 and 16, PCR primers for amplification of au-cat-PtacfadLfadD gene fragment

SEQ ID NO: 17, Nucleotide sequence of lcfA gene of Bacillus subtilis

SEQ ID NO: 18, Amino acid sequence of LcfA protein of Bacillus subtilis

SEQ ID NOS: 19 and 20, PCR primers for amplification of fadL gene fragment

SEQ ID NOS: 21 and 22, PCR primers for amplification of att-cat-Ptac fragment

SEQ ID NOS: 23 and 24, PCR primers for amplification of lcfA gene fragment

SEQ ID NOS: 25 and 26, PCR primers for amplification of att-cat-PtacfadLlcfA gene fragment

1. A method for producing an L-amino acid comprising:
culturing a bacterium belonging to the family Enterobacteriaceae and having an L-amino acid-producing ability in a medium comprising a fatty acid; and
collecting the L-amino acid from the medium,
wherein the bacterium is a bacterium introduced with the lcfA gene, and
the lcfA gene is a DNA selected from the group consisting of:
(A) a DNA encoding a protein comprising the amino acid sequence of SEQ ID NO: 18;
(B) a DNA encoding a protein comprising the amino acid sequence of SEQ ID NO: 18 including substitution, deletion, insertion, or addition of one or several amino acid residues, and having an activity of generating a fatty acyl-CoA from a long chain fatty acid, and taking it up through the inner membrane;
(C) a DNA comprising the nucleotide sequence of SEQ ID NO: 17; and
(D) a DNA hybridizable under stringent conditions with a nucleotide sequence complementary to the nucleotide sequence of SEQ ID NO: 17, or with a probe that can be prepared from the nucleotide sequence, and encoding a protein having an activity of generating a fatty acyl-CoA from a long chain fatty acid, and taking it up through the inner membrane.

2. The method according to claim 1, wherein the bacterium has been further modified so that fatty acid-assimilating ability thereof is increased.

3. The method according to claim 2, wherein the fatty acid-assimilating ability is increased by a method selected from the group consisting of:
(a) attenuating the expression of the fadR gene,
(b) enhancing the expression of a gene selected from the group consisting of fadL, fadE, fadD, fadB, fadA, and combinations thereof,
(c) enhancing the expression of the cyoABCDE operon, and
(d) combinations thereof.

4. The method according to claim 1, wherein the fatty acid is oleic acid.

5. The method according to claim 1, wherein the medium further comprises a carbon source in addition to the fatty acid.

6. The method according to claim 5, wherein the carbon source in addition to the fatty acid is glucose.

7. The method according to claim 1, wherein the L-amino acid is L-lysine.

8. The method according to claim 1, wherein the bacterium is an Escherichia bacterium.

9. The method according to claim 8, wherein the bacterium is Escherichia coli.
